(19) United States
(12) Patent Application Publication
(10) Pub. No.: US 2008/0234354 A1
(43) Pub. Date: Sep. 25, 2008

(54) Methods And Compositions For Controlling Body Weight And Appetite

(76) Inventors: Arnold S. LIPPA, New York, NY (US); Joseph W. Epstein, Monroe, NY (US); Joseph T. Tizzano, Holmdel, NJ (US); Anthony Basile, Hoboken, NJ (US)

Correspondence Address: Jeffrey J. King, Esq.;BLACK LOWE & GRAHAM PLLC, 701 Fifth Avenue, Suite 4800, Seattle, WA, 98104, US

(21) Appl. No.: 11/943,552

(22) Filed: Nov. 20, 2007

Related U.S. Application Data
(63) Continuation-in-part of application No. 11/603,974, filed on Nov. 21, 2006.

(51) Int. Cl. A61K 31/403; A61P 3/04; A61P 3/10

(52) U.S. Cl. 514/412

(57) ABSTRACT

The present invention provides novel compositions and methods for the controlling appetite and weight and/or treating obesity using a (+)-1-(3,4-dichlorophenyl)-3-azabicyclo[3.1.0]hexane or related compound. The invention also provides novel compositions and methods for treating or preventing disorders related to or complicated by excessive body weight or obesity, including coronary heart disease, osteoarthritis, osteoporosis, dislipidemias, gout, atherosclerosis, joint pain, sexual and fertility problems, respiratory problems, gall bladder disease, skin conditions, hypertension, diabetes, stroke, pulmonary embolism, sleep apnea, idiopathic intracranial hypertension, lower extremity venous stasis disease, gastro-esophageal reflux, urinary stress incontinence, metabolic syndrome, insulin resistance and cancer. The methods and compositions of the invention may employ a (+)-1-(3,4-dichlorophenyl)-3-azabicyclo[3.1.0]hexane or related compound alone, or in combination with a second anti-appetite or anti-obesity agent.

### RELATED APPLICATIONS

This application is a Continuation-In-Part of U.S. patent application Ser. No. 11/603,974, filed Nov. 21, 2006.

### TECHNICAL FIELD

The present invention relates to novel compositions and methods for controlling weight and appetite.

### BACKGROUND OF THE INVENTION

Sixty-Five percent (65%) of the U.S. population is overweight or obese, and there are over 300 million obese adults worldwide (Centers for Disease Control and Prevention. Prevalence of overweight and obesity among adults: United States, 1999-2002). Death rates escalate with increasing body weight. Among subjects whose body mass index (BMI) exceeds 30 kg/m², more than 50% of all-cause mortality is attributable to obesity-related conditions (Lee, JAMA 268:2045-2049, 1992). Obesity contributes to more than 300,000 deaths per year in the U.S., and ranks second only to smoking among preventable mortality causes (McGinnis, JAMA 270:2207-2212, 1993).

Total economic costs attributable to obesity were estimated at nearly $ 100 billion in 1998. $78.5 billion of these estimated costs were direct medical expenses. Obesity accounts for approximately 9.1% of total medical care costs, and obese individuals spend approximately 36% more on health services and 77% more on medications than normal-weight individuals. The cost of obesity to U.S. business in 1998 was estimated at $12.7 billion (Finkelstein E A, Fiebelkorn I C, Wang G. State-level estimates of annual medical expenditures attributable to obesity. Obesity Research. January 2004; 18-24).

Obesity is a well-established risk factor for coronary heart disease, osteoarthritis, gout, atherosclerosis, joint pain, sexual and fertility problems, respiratory problems, skin conditions, hypertension, diabetes, stroke, pulmonary embolism, sleep apnea, idiopathic intracranial hypertension, lower extremity venous stasis disease, gastro-esophageal reflux, urinary stress incontinence, and cancer. It also complicates chronic respiratory disease, osteoarthritis, osteoporosis, gall bladder disease, and dyslipidemia. In addition, obesity can contribute to psychological disorders such as depression and eating disorders.

The terms “hunger” and “satiety” are conventional terms in the art used to describe an individual's drive to obtain and ingest food. These neurophysiological responses are controlled in part by nerve connections between the stomach and duodenum and brain, as well as by circulating hormones that affect an individual's perceptions of hunger/satiety. Other factors that affect appetite include psychological factors, such as eating for pleasure, eating in a social context, and physical factors, such as blood sugar levels, dehydration, and physical activity.

While there are proposed genetic factors linked to obesity, the accumulation of body fat in most obese subjects is directly related to caloric intake. A small percentage of obese individuals have metabolic disorders in which they ingest few calories yet maintain excess body mass. However, even these weight conditions are attributable to ingestion of more calories than are expended, leading to sustained or increased body mass.

The drive to overeat is often related to self-control issues and aberrant psychological conditions, such as stress or depression. Many approaches to weight loss and obesity treatment involving psychological intervention, behavior modification, dietary change, pharmaceutical therapies, and/or surgery have been tried, with limited success. Psychological intervention, for example lifestyle programs that include cognitive-behavioral methods for modifying diet, physical activity, and psychological functioning, may be effective for producing gradual and moderate short-term weight losses. However, in most studies with extended follow-up, patients gradually return to baseline within a few years after treatment termination unless some form of maintenance program with sustained contact is implemented. Other behavioral modification treatments have been largely ineffective and associated with long-term recidivism rates exceeding 95% (NIH Technology Assessment Conference Panel, Ann. Intern. Med. 119:764-770, 1993).

Dietary change is the most commonly used weight loss strategy. Methods range from caloric restriction to changes in dietary proportions of fat, protein, and carbohydrate or the use of macronutrient substitutes. Weight loss at the end of relatively short-term programs can exceed 10 percent of initial body weight; however, there is a strong tendency to regain weight, with as much as two thirds of the weight lost regained within 1 year of completing the program and almost all regained by 5 years.

Surgical obesity treatments, such as gastric partitioning, jejunoileal bypass, and vagotomy, have been developed to treat severe obesity. (Greenway, Endocrinol. Metab. Clin. N. Amer. 25:1005-1027, 1996). Although these surgical procedures are somewhat more effective in the long run than the current pharmacological treatments, the acute risk-benefit ratio of invasive surgery and subsequent complications have reserved these procedures for morbidly obese patients having a body mass index >40 kg/m². (NIH Conference, Ann. Intern. Med. 115:956-961, 1991). Therefore, this approach is not an alternative for the majority of overweight and obese patients.

Another approach to treating obesity is the use of pharmaceutical agents. Pharmaceutical agents for treating obesity are generally divided into three groups: (1) drugs that decrease food intake, such as drugs that interfere with monoamine receptors, including noradrenergic receptors, serotonin receptors, dopamine receptors, and histamine receptors; (2) drugs that increase metabolism; and (3) drugs that increase thermogenesis or decrease fat absorption by inhibiting pancreatic lipase (Bray, 2000, Nutrition 16:953-960 and Leonhardt et al., 1999, Eur. J. Nutr. 38:1-13). Currently prescribed drugs for treating obesity include orlistat, which reportedly reduces the amount of dietary fat absorbed from the intestine; sibutramine, which reportedly suppresses appetite by inhibiting re-uptake of norepinephrine and serotonin; fenfluramine, d-fenfluramine and diethylpropion, which reportedly suppress appetite by releasing serotonin and inhibiting its re-uptake; and phentermine, which reportedly suppresses appetite by stimulating release of norepinephrine.

Despite this diverse assemblage of reportedly useful drug candidates for treating obesity, current drug therapies for weight reduction typically achieve no better than 5% to 10% decrease in body weight (National Task Force on the Prevention and Treatment of Obesity: Long-term pharmacotherapy in the Management of Obesity, JAMA 276:1907-15, 1996). Current obesity drugs also frequently have serious side effects, such as dizziness, headache, rapid pulse, palpitations, sleeplessness, hypertension, diarrhea, and intestinal cramping. For example, a combination of fenfluramine and phentermine, which reportedly produces a 15% to 20% reduction in body weight (F. Brenot et al., Appetite Suppressant Drugs and the Risk of Primary Pulmonary Hypertension, N. Engl. J. Med., 335:609-16, 1996), increases risk of heart valve damage and has reportedly contributed to numerous patient deaths. Another obesity drug, diethylpropion, has been linked to primary pulmonary hypertension. Other obesity medications, such as adderall (a combination of amphetamine and dextroamphetamine, mazindol and benzphetamine), show potential for addiction and are therefore not recommended for long term use.

In the United States alone, obesity increased from 12 percent of the population in 1991 to 17.9 percent in 1998, clearly demonstrating that the growing obesity epidemic is threatening the health of millions of individuals. (Mokdad A H, Serdula M K, Dietz W, Bowman B A, Marks J S, Koplan J P. The spread of the obesity epidemic in the United States, 1991-1998. JAMA 1999; 282:1519-22) Existing weight loss therapies fail to provide adequate benefit to many obese patients because of adverse side effects, contraindications or lack of lasting positive response (National Heart, Lung and Blood Institute, Clinical guidelines on the identification, evaluation, and treatment of overweight and obesity in adults: the evidence report, NIH Publication No. 98-4083, September 1998).

There is therefore an urgent need in the art for new and alternative tools and methods for controlling weight and appetite and treating obesity.

It is therefore an object of the present invention to provide methods and compositions for controlling weight gain.

It is also an object of the invention to provide methods and compositions for controlling appetite.

It is a further object of the invention to provide methods and compositions for stimulating weight loss.

It is an additional object of the present invention to provide methods and compositions for achieving sustained weight loss.

It is yet another object of the invention to provide methods and compositions for treating obesity.

### SUMMARY OF EXEMPLARY EMBODIMENTS OF THE INVENTION

The invention achieves these objects and satisfies additional objects and advantages by providing novel and surprisingly effective compositions and methods for controlling appetite, limiting or preventing weight gain, reducing caloric intake, and/or treating obesity in vertebrate subjects, typically mammalian subjects. The methods and

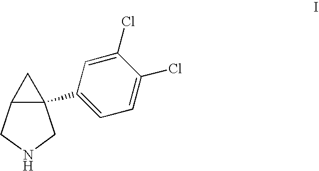

compositions of the invention employ surprisingly effective appetite-reducing and/or weight-controlling compounds, which are selected from (+)-1-(3,4-dichlorophenyl)-3-azabicyclo[3.1.0]hexanes of formula I, above, and related compounds and derivatives.

Useful forms of (+)-1-(3,4-dichlorophenyl)-3-azabicyclo[3.1.0]hexane within the formulations and methods of the invention include the compounds described herein, as well as their active pharmaceutically acceptable salts, polymorphs, solvates, hydrates, and/or prodrugs, and combinations thereof. Additional description relating to (+)-1-(3,4-dichlorophenyl)-3-azabicyclo[3.1.0]hexane for use within the formulations and is provided, for example, in U.S. patent application Ser. No. 11/442,743, filed May 25, 2006, U.S. patent application Ser. No. 10/466,457 filed Feb. 10, 2004, and PCT/US02/00845 filed Jan. 11, 2002, each of which disclosures is incorporated herein by reference.

In exemplary embodiments, the compositions and methods of the invention employ a (+)-1-(3,4-dichlorophenyl)-3-azabicyclo[3.1.0]hexane to: i) reduce appetite; ii) induce satiety; iii) reduce body weight; iv) limit or prevent weight gain and/or obesity; and/or v) treat or prevent one or more disease(s) or condition(s) associated with obesity, such as hypertension.

Subjects amenable for treatment using the formulations and methods of the invention include, but are not limited to, human and other mammalian subjects suffering from an appetite disorder, excess weight or obesity, and/or disorders related to or complicated by being overweight, including, but not limited to, coronary heart disease, osteoarthritis, osteoporosis, dislipidemias, gout, atherosclerosis, joint pain, sexual and fertility problems, respiratory problems, gall bladder disease, skin conditions, hypertension, diabetes, stroke, pulmonary embolism, sleep apnea, idiopathic intracranial hypertension, lower extremity venous stasis disease, gastro-esophageal reflux, urinary stress incontinence, metabolic syndrome, insulin resistance and cancer.

These and other subjects are effectively treated prophylactically and/or therapeutically by administering to the subject an effective amount of a (+)-1-(3,4-dichlorophenyl)-3-azabicyclo[3.1.0]hexane or related compound as described herein sufficient to suppress appetite, reduce body weight, decrease body fat, and/or decrease weight gain in the subject. As noted above, the methods and formulations of the present invention may employ (+)-1-(3,4-dichlorophenyl)-3-azabicyclo[3.1.0]hexane in a variety of forms including pharmaceutically acceptable salts, polymorphs, solvates, hydrates and/or prodrugs or combinations thereof. (+)-1-(3,4-dichlorophenyl)-3-azabicyclo[3.1.0]hexane is employed as an illustrative embodiment of the invention in the examples herein below.

Within additional aspects of the invention, combinatorial formulations and methods are provided which employ an effective amount of (+)-1-(3,4-dichlorophenyl)-3-azabicyclo[3.1.0]hexane and one or more secondary or adjunctive therapeutic agent(s) that are combinatorial formulated or coordinately administered with (+)-1-(3,4-dichlorophenyl)-3-azabicyclo[3.1.0]hexane to suppress appetite, reduce body weight, and/or decrease weight gain. Exemplary combinatorial formulations and coordinate treatment methods in this context employ (+)-1-(3,4-dichlorophenyl)-3-azabicyclo[3.1.0]hexane in combination with one or more additional secondary or adjunctive active agent(s) that are combinatorially formulated or coordinately administered with the (+)-1-(3,4-dichlorophenyl)-3-azabicyclo[3.1.0]hexane to yield an effective anti-obesity response. The secondary or adjunctive therapeutic agents used in conjunction with (+)-1-(3,4-dichlorophenyl)-3-azabicyclo[3.1.0]hexane in these embodiments may possess direct or indirect effects to suppress appetite, reduce body weight, and/or decrease weight gain alone or in combination with (+)-1-(3,4-dichlorophenyl)-3-azabicyclo[3.1.0]hexane or may exhibit other useful adjunctive therapeutic activity in combination with (+)-1-(3,4-dichlorophenyl)-3-azabicyclo[3.1.0]hexane. Useful secondary or adjunctive agents in these combinatorial formulations and coordinate treatment methods include, for example, other appetite-suppressing agents or anti-obesity agents including, but not limited to, insulin sensitizers, biguanides, protein tyrosine phosphatase-1B (PTP-1B) inhibitors, dipeptidyl peptidase IV (DP-IV) inhibitors, insulin or insulin mimetics, sulfonylureas, cholesterol lowering agents, sequestrants, nicotinyl alcohol, nicotinic acid, PPARα agonists, PPARα/γ dual agonists, carbonic anhydrase inhibitors, inhibitors of cholesterol absorption, acyl CoA:cholesterol acyltransferase inhibitors, anti-oxidants, anti-obesity compounds, neuropeptide Y5 inhibitors, β₃ adrenergic receptor agonists, ileal bile acid transporter inhibitors, anti-inflammatories and cyclo-oxygenase 2 selective inhibitors. Adjunctive therapies may also be used including, but not limited, physical treatments such as changes in diet, psychological counseling, behavior modification, exercise and surgery including, but not limited to, gastric partitioning procedures, jejunoileal bypass, stomach stapling, gastric bands, vertical banded gastroplasty, laparoscopic gastric banding, roux-en-Y gastric bypass, biliopancreatic bypass procedures and vagotomy.

The foregoing and other objects, features, aspects and advantages of the present invention will become more apparent from the following detailed description of the invention.

### BRIEF DESCRIPTION OF DRAWINGS

FIG. 1 is a dose-response graph depicting the effect of (+)-1-(3,4-dichlorophenyl)-3-azabicyclo[3.1.0]hexane (DOV 21947) on the body weight of male DIO rats 18 hours post treatment, compared to AM 251 dexfenfluramine.

FIG. 2 is a graph depicting the change in body weight of male DIO rats following 14 days of treatment with (+)-1-(3,4-dichlorophenyl)-3-azabicyclo[3.1.0]hexane, in comparison with AM251, sibutramine and dexfenfluramine.

FIG. 3 consists of two graphs depicting the effect of (+)-1-(3,4-dichlorophenyl)-3-azabicyclo[3.1.0]hexane (DOV 21947) and reference agents AM251, sibutramine and dexfenfluramine on the cumulative food intake, and cumulative feeding efficiency of male DIO rats following 14 days of administration.

FIG. 4 consists of two graphs, the first depicting the change in lean, fat and total body mass of male DIO rats, and the second showing the specific changes in white adipose tissue (WAT) depots, in both cases following 14 days of treatment with (+)-1-(3,4-dichlorophenyl)-3-azabicyclo[3.1.0]hexane (DOV 21947) and reference agents AM251, sibutramine and dexfenfluramine.

FIG. 5 consists of three graphs, the first depicting the change in body weight, the second the change in cumulative food intake, and the third shows the change in total fat mass following 21-24 days of treatment with (+)-1-(3,4-dichlorophenyl)-3-azabicyclo[3.1.0]hexane (DOV 21947).

FIG. 6 is a graph depicting the change in plasma triglyceride levels of male DIO rats following 14 days of treatment with (+)-1-(3,4-dichlorophenyl)-3-azabicyclo[3.1.0]hexane, in comparison with AM251, sibutramine and dexfenfluramine.

FIG. 7 is a graph showing changes in body weight of male rats administered 0, 10, 25, or 60 mg/kg/day respectively of (+)-1-(3,4-dichlorophenyl)-3-azabicyclo[3.1.0]hexane orally for 13 weeks.

FIG. 8 is a graph showing changes in body weight of female rats administered 0, 10, 25, or 60 mg/kg/day respectively of (+)-1-(3,4-dichlorophenyl)-3-azabicyclo[3.1.0]hexane orally for 13 weeks.

FIG. 9 is a graph depicting cumulative changes in body weight of male rats administered 0, 10, 25, or 60 mg/kg/day respectively of (+)-1-(3,4-dichlorophenyl)-3-azabicyclo[3.1.0]hexane orally for 13 weeks.

FIG. 10 is a graph showing cumulative changes in body weight of female rats administered 0, 10, 25, or 60 mg/kg/day respectively of (+)-1-(3,4-dichlorophenyl)-3-azabicyclo[3.1.0]hexane orally for 13 weeks.

FIG. 11 is a graph showing changes in body weight of male dogs administered 0, 2.0, 6.0, or 20 mg/kg/day respectively of (+)-1-(3,4-dichlorophenyl)-3-azabicyclo[3.1.0]hexane orally for 13 weeks.

FIG. 12 is a graph demonstrating changes in body weight of female dogs administered 0, 2.0, 6.0, or 20 mg/kg/day respectively of (+)-1-(3,4-dichlorophenyl)-3-azabicyclo[3.1.0]hexane orally for 13 weeks.

FIG. 13 is a graph showing cumulative changes in body weight of male dogs administered 0, 2.0, 6.0, or 20 mg/kg/day respectively of (+)-1-(3,4-dichlorophenyl)-3-azabicyclo[3.1.0]hexane orally for 13 weeks.

FIG. 14 is a graph demonstrating cumulative changes in body weight of male dogs administered 0, 2.0, 6.0, or 20 mg/kg/day respectively of (+)-1-(3,4-dichlorophenyl)-3-azabicyclo[3.1.0]hexane orally for 13 weeks.

FIG. 15 is a graph depicting the change in body weight of humans following a 2 month, ascending dose treatment schedule with 14 days of treatment with (+)-1-(3,4-dichlorophenyl)-3-azabicyclo[3.1.0]hexane (DOV 21947), on the last day of treatment (left side), and 7 days after the last day of treatment (right side).

FIG. 16 is a graph depicting the change in body mass index of humans following a 2 month, ascending dose treatment schedule with 14 days of treatment with (+)-1-(3,4-dichlorophenyl)-3-azabicyclo[3.1.0]hexane, on the last day of treatment (left side), and 7 days after the last day of treatment (right side).

FIG. 17 is a graph depicting the change in plasma triglyceride levels of humans following a 2 month, ascending dose treatment schedule with 14 days of treatment with (+)-1-(3,4-dichlorophenyl)-3-azabicyclo[3.1.0]hexane, on the last day of treatment (left side), and 7 days after the last day of treatment (right side).

### DETAILED DESCRIPTION OF EXEMPLARY EMBODIMENTS OF THE INVENTION

The instant invention provides novel compositions and methods for controlling appetite or weight, and/or treating obesity using a (+)-1-(3,4-dichlorophenyl)-3-azabicyclo[3.1.0]hexane or related compound. In various embodiments, the methods and compositions of the invention are effective for decreasing appetite, reducing weight, decreasing body fat, increasing lean muscle mass ratio, lowering body mass and/or reducing symptoms and diseases associated with or complicated by obesity.

Formulations and methods of the invention employ (+)-1-(3,4-dichlorophenyl)-3-azabicyclo[3.1.0]hexane and its derivatives for the treatment of obesity. Within these formulations and methods, (+)-1-(3,4-dichlorophenyl)-3-azabicyclo[3.1.0]hexane may be provided in any of a variety of forms, including any pharmaceutically acceptable salt, solvate, hydrate, polymorph, or prodrug of a (+)-1-(3,4-dichlorophenyl)-3-azabicyclo[3.1.0]hexane, and/or combinations thereof. As described herein, (+)-1-(3,4-dichlorophenyl)-3-azabicyclo[3.1.0]hexane and related compounds are effective to treat mammalian subjects suffering from excess appetite, abnormal body weight, and/or obesity, as well as disorders related to or complicated by being overweight, including, but not limited to, coronary heart disease, osteoarthritis, osteoporosis, dislipidemias, gout, atherosclerosis, joint pain, sexual and fertility problems, respiratory problems, gall bladder disease, skin conditions, hypertension, diabetes, stroke, pulmonary embolism, sleep apnea, idiopathic intracranial hypertension, lower extremity venous stasis disease, gastro-esophageal reflux, urinary stress incontinence, metabolic syndrome, insulin resistance and cancer.

Within the methods and compositions of the invention, (+)-1-(3,4-dichlorophenyl)-3-azabicyclo[3.1.0]hexane compounds of formula I, above, or related compounds or derivatives as disclosed herein, are effectively formulated and administered as anti-appetite or anti-obesity agents for treating excessive appetite, obesity and/or related disorders. In exemplary embodiments, (+)-1-(3,4-dichlorophenyl)-3-azabicyclo[3.1.0]hexane is demonstrated for illustrative purposes to be an anti-obesity effective agent in pharmaceutical formulations alone or in combination with one or more secondary or adjunctive agents. The present disclosure further provides additional, pharmaceutically acceptable (+)-1-(3,4-dichlorophenyl)-3-azabicyclo[3.1.0]hexane compounds including complexes, derivatives, salts, solvates, polymorphs and prodrugs of the compounds disclosed herein, and combinations thereof, which are effective as anti-obesity therapeutic agents within the methods and compositions of the invention.

A broad range of mammalian subjects, including human subjects, are amenable for treatment using the formulations and methods of the invention. These subjects include, but are not limited to, human and other mammalian subjects suffering from excess weight including obesity and disorders related to or complicated by being overweight, including, but not limited to, coronary heart disease, osteoarthritis, osteoporosis, dislipidemias, gout, atherosclerosis, joint pain, sexual and fertility problems, respiratory problems, gall bladder disease, skin conditions, hypertension, diabetes, stroke, pulmonary embolism, sleep apnea, idiopathic intracranial hypertension, lower extremity venous stasis disease, gastro-esophageal reflux, urinary stress incontinence, metabolic syndrome, insulin resistance and cancer. As used herein, the term “obesity” includes both excess body weight and excess adipose tissue mass in an animal. An obese human is an individual having a body mass index of ≧30 kg/m².

The (+)-1-(3,4-dichlorophenyl)-3-azabicyclo[3.1.0]hexanes used in the methods and compositions of the present invention are represented by the structural formula I.

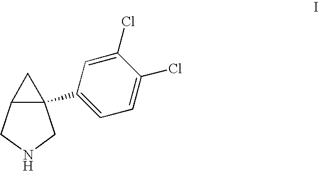

It will be appreciated by those skilled in the art that the compound of Formula I contains at least one chiral center and is presented in an enantiomeric form. The enantiomers of (±)-1-(3,4-dichlorophenyl)-3-azabicyclo[3.1.0]hexane, particularly the (+)-1-(3,4-dichlorophenyl)-3-azabicyclo[3.1.0]hexane of Formula I, may be resolved by methods known to those skilled in the art, including, but not limited to, formation of diastereoisomeric salts or complexes which may be separated by methods including, but not limited to: crystallization; gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, for example, silica with a bound chiral ligand or in the presence of a chiral solvent. Alternatively, specific enantiomers may be synthesized by asymmetric synthesis using optically active reagents, substrates, catalysts or solvents, or by converting one enantiomer to the other by asymmetric transformation. In one exemplary embodiment, (+)-1-(3,4-dichlorophenyl)-3-azabicyclo[3.1.0]hexane substantially free of a corresponding (−)-1-(3,4-dichlorophenyl)-3-azabicyclo[3.1.0]hexane enantiomer can be obtained from (±)-1-(3,4-dichlorophenyl)-3-azabicyclo[3.1.0]hexane using chiral chromatographic methods, such as high-performance liquid chromatography (“HPLC”) with a suitable, e.g., chiral, column. (±)-1-(3,4-dichlorophenyl)-3-azabicyclo[3.1.0]hexane is obtainable using methods disclosed in U.S. Pat. No. 4,435,419 to Epstein et al., incorporated herein by reference in its entirety. In another embodiment, (+)-1-(3,4-dichlorophenyl)-3-azabicyclo[3.1.0]hexane can be obtained by resolving (±)-1-(3,4-dichlorophenyl)-3-azabicyclo[3.1.0]hexane using a chiral polysaccharide stationary phase and an organic eluent. Preferably, the polysaccharide is starch or a starch derivative. Advantageously, a chiral HPLC column can be used, for example, a CHIRALPAK AD column (manufactured by Daicel and commercially available from Chiral Technologies, Inc., Exton, Pa.) more preferably a 1 cm×25 cm CHIRALPAK AD HPLC column. The preferred eluent is a hydrocarbon solvent adjusted in polarity with a miscible polar organic solvent. Preferably, the organic eluent contains a non-polar, hydrocarbon solvent present in about 95% to about 99.5% (volume/volume) and a polar organic solvent present in about 5% to about 0.5% (volume/volume). In a preferred embodiment, the hydrocarbon solvent is hexane and the miscible polar organic solvent is isopropylamine. As used herein, the term “substantially free of its corresponding (−)-enantiomer” means approximately 5% or less w/w of the corresponding (−)-enantiomer, preferably no more than about 2% w/w of the corresponding (−)-enantiomer, more preferably no more than about 1% w/w of the corresponding (−)-enantiomer. In a further embodiment, an alternative chromatographic procedure, known as simulated moving bed (SMB) chromatography can be employed for the resolution of (±)-1-(3,4-dichlorophenyl)-3-azabicyclo[3.1.0]hexane. SMB is increasingly becoming the method of choice for large-scale enantiomer separation in the pharmaceutical industry (See Chemical and Engineering News, Vol. 79, No. 20, p. 47 (2001)). In yet another embodiment, the resolution of racemic (±)-1-(3,4-dichlorophenyl)-3-azabicyclo[3.1.0]hexane to obtain (+)-1-(3,4-dichlorophenyl)-3-azabicyclo[3.1.0]hexane can be achieved via the use of optically active resolving acids via the formation of, and subsequent separation of, the resulting diasteromeric salts. Commonly employed chiral acids for this purpose include: tartaric and O-acyl tartaric acids, mandelic acid and O-substituted mandelic acids, 1,1′-binaphthyl-2,2′-diyl hydrogen phosphate, camphoric acid, camphor sulfonic acid, and other readily-available optically active acids (both commercially available and readily synthesized).

Within the methods and compositions of the invention, (+)-1-(3,4-dichlorophenyl)-3-azabicyclo[3.1.0]hexane is effectively formulated or administered to treat weight gain, obesity, and/or obesity related conditions in mammals. In exemplary embodiments, (+)-1-(3,4-dichlorophenyl)-3-azabicyclo[3.1.0]hexane is shown to be an effective agent in pharmaceutical formulations and methods. It is further apparent from the present disclosure that additional pharmaceutically acceptable (+)-1-(3,4-dichlorophenyl)-3-azabicyclo[3.1.0]hexane compounds, complexes, salts, polymorphs, solvates, hydrates and/or prodrugs, or combinations thereof will be comparably effective in treating weight gain and obesity within the methods and compositions of the invention.

Polymorphs are compounds with identical chemical structure but different internal structures. Additionally, many pharmacologically active organic compounds regularly crystallize incorporating second, foreign molecules, especially solvent molecules, into the crystal structure of the principal pharmacologically active compound forming pseudopolymorphs. When the second molecule is a solvent molecule, the pseudopolymorphs can also be referred to as solvates. All of these additional forms of (+)-1-(3,4-dichlorophenyl)-3-azabicyclo[3.1.0]hexane are likewise useful and considered to be within the anti-appetite and anti-obesity methods and formulations of the invention.

Obesity treating compositions of the invention typically comprise an amount of (+)-1-(3,4-dichlorophenyl)-3-azabicyclo[3.1.0]hexane of Formula I, its pharmaceutically acceptable salts, polymorphs, solvates, hydrates, and/or prodrugs, or combinations thereof, which is effective for controlling appetite and/or treatment or prevention of weight gain or obesity, or complications and related conditions thereof, in a mammalian subject. Typically, an anti-appetite or anti-obesity effective amount, of a (+)-1-(3,4-dichlorophenyl)-3-azabicyclo[3.1.0]hexane compound or (+)-1-(3,4-dichlorophenyl)-3-azabicyclo[3.1.0]hexane related or derivative compound of Formula I will comprise an amount of the active compound which is effective, in a single or multiple unit dosage form, over a specified period of administration, to measurably reduce appetite or caloric intake, or to alleviate one or more symptoms of obesity or a related condition in the subject. The active compound(s) may be optionally formulated with a pharmaceutically acceptable carrier and/or various excipients, vehicles, stabilizers, buffers, preservatives, etc.

The amount, timing and mode of delivery of compositions of the invention comprising an anti-obesity effective amount of a (+)-1-(3,4-dichlorophenyl)-3-azabicyclo[3.1.0]hexane compound or derivative compound of Formula I will be routinely adjusted on an individual basis, depending on such factors as weight, age, gender, and condition of the individual, the acuteness or severity of the appetite or weight disorder, whether the administration is prophylactic or therapeutic, and on the basis of other factors known to effect drug delivery, absorption, pharmacokinetics, half-life, etc.

An effective dose or multi-dose treatment regimen for the instant anti-obesity formulations will ordinarily be selected to approximate a minimal dosing regimen that is necessary and sufficient to substantially prevent or alleviate obesity and related conditions in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years.

An “effective amount,” “therapeutic amount,” “therapeutic effective amount,” or “effective dose” is an amount or dose sufficient to elicit a desired pharmacological or therapeutic effect in a mammalian subject—for example to achieve a measurable reduction in appetite, caloric intake, body weight, body fat or percentage of body fat relative to lean muscle mass. Therapeutic efficacy can alternatively be demonstrated by a decrease in food intake or weight gain; or by a decrease in weight, body fat, percentage of body fat, circumference of body parts; improvement of the waist/hip ratio; movement on a height/weight chart; or by altering the nature, recurrence, or duration of symptoms associated with obesity including respiratory ailments; shortness of breath; joint pain; and muscle aches; and altering the nature, recurrence, severity or duration of conditions which are more common in, associated with, or complicated by being overweight and obese, including but not limited to, coronary heart disease, osteoarthritis, osteoporosis, dislipidemias, gout, atherosclerosis, sexual and fertility problems, respiratory problems, gall bladder disease, skin conditions, hypertension, diabetes, stroke, pulmonary embolism, sleep apnea, idiopathic intracranial hypertension, lower extremity venous stasis disease, gastro-esophageal reflux, urinary stress incontinence, metabolic syndrome, insulin resistance and cancer.

Therapeutic effectiveness may be determined, for example, through a change in body fat as determined by body fat measurements. Body fat measurements may be determined by a variety of means including, but not limited to, determinations of skinfold thickness, bioelectrical impedance, underwater weighing, DEXA scans, measurement on a scale or calculation of body mass index (BMI).

Percentages of weight due to body fat for normal men are between 10-20%. In athletes, the normal range is between 6-10%. In women, the normal range is between 15-25% and in athletic women it is between 10-15%. Effective amounts of the compounds of the present invention will decrease body fat percentages from above 20-25%. Effective amounts may also decrease body fat percentages to within the normal ranges for that individual. Effectiveness may also be demonstrated by a 2-50%, 10-40%, 15-30%, 20-25% decrease in body fat.

Skinfold measurements measure subcutaneous fat located directly beneath the skin by grasping a fold of skin and subcutaneous fat between the thumb and forefinger and pulling it away from the underlying muscle tissue. The thickness of the double layer of skin and subcutaneous tissue is then read with a caliper. The five most frequently measured sites are the upper arm, below the scapula, above the hip bone, the abdomen, and the thigh. Skinfold measurements are used to determine relative fatness, changes in physical conditioning programs, and the percentage of body fat in desirable body weight. Effective amounts of (+)-1-(3,4-dichlorophenyl)-3-azabicyclo[3.1.0]hexane compounds will decrease body fat percentages by 2-50%, 10-40%, 15-30%, 20-25%, 30-40% or more.

Body fat percentages can also be determined by body impedance measurements. Body impedance is measured when a small electrical signal is passed through the body carried by water and fluids. Impedance is greatest in fat tissue, which contains only 10-20% water, while fat-free mass, which contains 70-75% water, allows the signal to pass much more easily. By using the impedance measurements along with a person's height, weight, and body type (gender, age, fitness level), it is possible to calculate the percentage of body fat, fat-free mass, hydration level, and other body composition values. Effective amounts of (+)-1-(3,4-dichlorophenyl)-3-azabicyclo[3.1.0]hexane compounds will decrease body fat percentages by 2-50%, 10-40%, 15-30%, 20-25%, 30-40% or more.

Hydrostatic or underwater weighing is another method for determining lean muscle mass and body fat percentages. It is based upon the application of the Archimedes principle, and requires weighing the subject on land, repeated weighing under water, and an estimation of air present in the lungs of the subject using gas dilution techniques. To perform the analysis, an individual is weighed as normal. The subject, in minimal clothing, then sits on a special seat, expels all air from the lungs and is lowered into a tank until all body parts are emerged. Underwater weight is then determined. Body density is then determined using the following calculation: Body density=Wa/(((Wa−Ww)/Dw)−(RV+100 cc)), where Wa=body weight in air (kg), Ww=body weight in water (kg), Dw=density of water, RV=residual lung volume, and 100 cc is the correction for air trapped in the gastrointestinal tract.

DEXA, or dual energy x-ray absorptiometry scans determine whole body as well as regional measurements of bone mass, lean mass, and fat mass. Total fat mass is expressed in kg and as a percentage of body mass. These are calculated by integrating the measurements for the whole body and different automatic default regions such as arms, trunk, and legs.

Body fat percentages may further be determined by air displacement plethysmography. Air displacement plethysmography determines the volume of a subject to be measured by measuring the volume of air displaced by the subject in an enclosed chamber. The volume of air in the chamber is calculated through application of Boyle's Law and/or Poisson's Law to conditions within the chamber. More particularly, in the most prevalent method of air displacement plethysmography used for measuring human body composition (such as disclosed in U.S. Pat. No. 4,369,652, issued to Gundlach, and U.S. Pat. No. 5,105,825, issued to Dempster), volume perturbations of a fixed frequency of oscillation are induced within a measurement chamber, which perturbations lead to pressure fluctuations within the chamber. The amplitude of the pressure fluctuations is determined and used to calculate the volume of air within the chamber using Boyle's Law (defining the relationship of pressure and volume under isothermal conditions) or Poisson's law (defining the relationship of pressure and volume under adiabatic conditions). Body volume is then calculated indirectly by subtracting the volume of air remaining inside the chamber when the subject is inside from the volume of air in the chamber when it is empty. Once the volume of the subject is known, body composition can be calculated based on the measured subject volume, weight of the subject, and subject surface area (which, for human subjects, is a function of subject weight and subject height), using known formulas defining the relationship between density and human fat mass.

Therapeutic effectiveness of (+)-1-(3,4-dichlorophenyl)-3-azabicyclo[3.1.0] treatment according to the invention may further be demonstrated, for example, through a change in body mass index. Body Mass Index (BMI) has been recognized by the U.S. Department of Health as a reference relationship between a person's height and weight and can be used to determine when extra weight above an average or normal weight range for a person of a given height can translate into and signal increased probability for additional health risks for that person. While BMI does not directly measure percent of body fat, higher BMIs are usually associated with an increase in body fat, and thus excess weight. A desired BMI range is from about 18 kg/m² to about 24 kg/m², wherein a person is considered to have a healthful weight for the person's height and is neither overweight nor underweight. A person with a BMI above 24 kg/m², such as from about 25 kg/m² to about 30 kg/m², is considered to be overweight, and a person with a BMI above about 30 kg/m² is considered to be obese. A person with a BMI above about 40 kg/m² is considered to be morbidly obese. In another aspect, an individual who has a BMI in the range of about 25 kg/m² to about 35 kg/m², and has a waist size of over 40 inches for a man and over 35 inches for a woman, is considered to be at especially high risk for health problems. Effectiveness of (+)-1-(3,4-dichlorophenyl)-3-azabicyclo[3.1.0] compounds may be demonstrated by a reduction in the body mass index from a range between 40 kg/m² to about 30 kg/m² to 25 kg/m² to about 24 kg/m². A compound of the present invention may also reduce BMI from a range above 30 kg/m² to a range between 30 kg/m² to 25 kg/m² and more preferably to about 24 kg/m². Effectiveness may further be demonstrated by a decrease in body weight from 2-50%, 10-40%, 15-30%, 20-25%. Effectiveness may additionally be demonstrated by a decrease in BMI by 2-50%, 10-40%, 15-30%, 20-25%, 30-40% or more. Effective amounts of (+)-1-(3,4-dichlorophenyl)-3-azabicyclo[3.1.0]hexane compounds will lower an individual's BMI to within about 18 kg/m² to about 24 kg/m².

Therapeutic effectiveness of (+)-1-(3,4-dichlorophenyl)-3-azabicyclo[3.1.0]hexane compounds of the present invention may also be determined by changes in the waist/hip ratio. The waist/hip ratio is determined by dividing the circumference of the waist by the circumference of the hip. Women should have a waist/hip ratio of 0.8 or less and men should have a waist/hip ratio of 0.95 or less. Effective amounts of (+)-1-(3,4-dichlorophenyl)-3-azabicyclo[3.1.0]hexane compounds will lower the waist/hip ratio by about 2-50%, 10-40%, 15-30%, 20-25% or more. The waist/hip ratio of a female subject may be lowered to 0.8 or less and the ratio of a male subject to a ratio of 0.95 or less.

Therapeutic effectiveness of (+)-1-(3,4-dichlorophenyl)-3-azabicyclo[3.1.0]hexane compounds of the present invention may also be determined by a decrease in weight of the subject as determined by a standard scale. Effective amounts of (+)-1-(3,4-dichlorophenyl)-3-azabicyclo[3.1.0]hexane compounds will decrease weight by about 2-50%, 10-40%, 15-30%, 20-25% or more.

Therapeutic effectiveness of (+)-1-(3,4-dichlorophenyl)-3-azabicyclo[3.1.0]hexane compounds of the present invention may also be determined by a decrease in caloric intake. Caloric intake may be determined by any method known to those skilled in the art including, but not limited to, food intake diaries and food histories. Effective amounts of (+)-1-(3,4-dichlorophenyl)-3-azabicyclo[3.1.0]hexane compounds will decrease caloric intake by about 2-50%, 10-40%, 15-30%, 20-25% or more.

Following administration of the (+)-1-(3,4-dichlorophenyl)-3-azabicyclo[3.1.0]hexane composition according to the formulations and methods of the invention, test subjects will exhibit a 5%, 10%, 20%, 30%, 50% or greater reduction, up to a 75-90%, or 95% or greater, reduction, in one or more symptoms associated with obesity, including weight, as compared to placebo-treated or other suitable control subjects. Test subjects may also exhibit a 10%, 20%, 30%, 50% or greater reduction, up to a 75-90%, or 95% or greater, reduction, in the symptoms of one or more conditions associated with or complicated by obesity including, but not limited to, coronary heart disease, osteoarthritis, osteoporosis, dislipidemias, gout, atherosclerosis, joint pain, sexual and fertility problems, respiratory problems, gall bladder disease, skin conditions, hypertension, diabetes, stroke, pulmonary embolism, sleep apnea, idiopathic intracranial hypertension, lower extremity venous stasis disease, gastro-esophageal reflux, urinary stress incontinence, metabolic syndrome, insulin resistance and cancer.

Therapeutically effective amounts, and dosage regimens, of (+)-1-(3,4-dichlorophenyl)-3-azabicyclo[3.1.0]hexane and its derivative compositions, including pharmaceutically effective salts, solvates, hydrates, polymorphs or prodrugs thereof, will be readily determinable by those of ordinary skill in the art, often based on routine clinical or patient-specific factors.

The pharmaceutical compositions of the present invention may be administered by any means that achieves the intended therapeutic or prophylactic purpose. Suitable routes of administration for obesity treating compositions of the invention comprising (+)-1-(3,4-dichlorophenyl)-3-azabicyclo[3.1.0]hexane include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Within additional aspects of the invention, combinatorial formulations and coordinate administration methods are provided which employ an effective amount of one or more (+)-1-(3,4-dichlorophenyl)-3-azabicyclo[3.1.0]hexane compositions, including pharmaceutically effective salts, solvates, hydrates, polymorphs or prodrugs thereof, and one or more additional active agent(s) that is/are combinatorially formulated or coordinately administered with the (+)-1-(3,4-dichlorophenyl)-3-azabicyclo[3.1.0]hexane or its derivative composition-yielding an effective formulation or method to modulate, alleviate, treat or prevent obesity in a mammalian subject. Exemplary combinatorial formulations and coordinate treatment methods in this context employ a (+)-1-(3,4-dichlorophenyl)-3-azabicyclo[3.1.0]hexane composition in combination with one or more additional or adjunctive therapeutic agents. Such additional or adjunctive therapeutic agents may be appetite suppressants or anti-obesity agents, including, but not limited to, insulin sensitizers including PPARγ agonists such as the glitazones (e.g. troglitazone, pioglitazone, englitazone, MCC-555, rosiglitazone); biguanides such as metformin and phenformin; protein tyrosine phosphatase-1B (PTP-1B) inhibitors; dipeptidyl peptidase IV (DP-IV) inhibitors; insulin or insulin mimetics; sulfonylureas such as tolbutamide and glipizide; α-glucosidase inhibitors (such as acarbose); cholesterol lowering agents such as HMG-CoA reductase inhibitors (lovastatin, simvastatin, pravastatin, fluvastatin, atorvastatin, rivastatin, itavastatin, ZD-4522 and other statins); sequestrants (cholestyramine, colestipol, and dialkylaminoalkyl derivatives of a cross-linked dextran); nicotinyl alcohol, nicotinic acid or a salt thereof; PPARα agonists such as fenofibric acid derivatives (gemfibrozil, clofibrate, fenofibrate and bezafibrate); PPARα/γ dual agonists, such as KRP-297; inhibitors of cholesterol absorption, such as, for example, beta-sitosterol; acyl CoA:cholesterol acyltransferase inhibitors, such as, for example, avasimibe; anti-oxidants, such as probucol; anti-obesity compounds such as, for example, fenfluramine, dexfenfluramine, phentiramine, sulbitramine, orlistat, diethylpropion, adderall, mazindol, and benzphetamine; neuropeptide Y5 inhibitors, and β₃ adrenergic receptor agonists; an ileal bile acid transporter inhibitor; and agents intended for use in inflammatory conditions such as aspirin, non-steroidal anti-inflammatory drugs, glucocorticoids, azulfidine, and cyclo-oxygenase 2 selective inhibitors. (+)-1-(3,4-dichlorophenyl)-3-azabicyclo[3.1.0]hexane may also be used in conjunction with physical treatments such as changes in diet, behavior modification, psychological counseling, exercise and surgery including, but not limited to, gastric partitioning procedures, jejunoileal bypass, stomach stapling, gastric bands, vertical banded gastroplasty, laparoscopic gastric banding, roux-en-Y gastric bypass, biliopancreatic bypass procedures and vagotomy.

In certain embodiments the invention provides combinatorial anti-obesity formulations comprising a (+)-1-(3,4-dichlorophenyl)-3-azabicyclo[3.1.0]hexane and one or more adjunctive agent(s) having weight loss or appetite suppressant activity. Within such combinatorial formulations, (+)-1-(3,4-dichlorophenyl)-3-azabicyclo[3.1.0]hexane and the adjunctive agent(s) having anti-obesity activity will be present in a combined formulation in effective amounts, alone or in combination. In exemplary embodiments, a (+)-1-(3,4-dichlorophenyl)-3-azabicyclo[3.1.0]hexane and a non-a (+)-1-(3,4-dichlorophenyl)-3-azabicyclo[3.1.0]hexane anti-obesity agent(s) will each be present in an anti-obesity amount (i.e., in singular dosage which will alone elicit a detectable anti-hyperlipidemia response in the subject). Alternatively, the combinatorial formulation may comprise one or both of the a (+)-1-(3,4-dichlorophenyl)-3-azabicyclo[3.1.0]hexane and non-a (+)-1-(3,4-dichlorophenyl)-3-azabicyclo[3.1.0]hexane e agents in sub-therapeutic singular dosage amount(s), wherein the combinatorial formulation comprising both agents features a combined dosage of both agents that is collectively effective in eliciting an anti-obesity response. Thus, one or both of the a (+)-1-(3,4-dichlorophenyl)-3-azabicyclo[3.1.0]hexane e and non-a (+)-1-(3,4-dichlorophenyl)-3-azabicyclo[3.1.0]hexane e agents may be present in the formulation, or administered in a coordinate administration protocol, at a sub-therapeutic dose, but collectively in the formulation or method they elicit a detectable anti-obesity response in the subject.

To practice the coordinate administration methods of the invention, a (+)-1-(3,4-dichlorophenyl)-3-azabicyclo[3.1.0]hexane compound is administered, simultaneously or sequentially, in a coordinate treatment protocol with one or more of the secondary or adjunctive therapeutic agents contemplated herein. The coordinate administration may be done simultaneously or sequentially in either order, and there may be a time period while only one or both (or all) active therapeutic agents, individually and/or collectively, exert their biological activities. A distinguishing aspect of all such coordinate treatment methods is that the (+)-1-(3,4-dichlorophenyl)-3-azabicyclo[3.1.0]hexane compound exert at least some detectable obesity modulating activity, and/or elicit a favorable clinical response, which may or may not be in conjunction with a secondary clinical response provided by the secondary therapeutic agent. Often the coordinate administration of a (+)-1-(3,4-dichlorophenyl)-3-azabicyclo[3.1.0]hexane compound with a secondary therapeutic agent as contemplated herein will yield an enhanced therapeutic response beyond the therapeutic response elicited by either or both the (+)-1-(3,4-dichlorophenyl)-3-azabicyclo[3.1.0]hexane compound and/or secondary therapeutic agent alone.

The amount, timing and mode of delivery of compositions of the invention comprising an effective amount of a (+)-1-(3,4-dichlorophenyl)-3-azabicyclo[3.1.0]hexane composition will be routinely adjusted on an individual basis, depending on such factors as weight, age, gender, and condition of the individual, the severity of the obesity or related symptoms, whether the administration is prophylactic or therapeutic, and on the basis of other factors known to effect drug delivery, absorption, pharmacokinetics, including, but not limited to, half-life, and efficacy. The precise dose to be employed will also depend on the route of administration, and the seriousness of the disease or disorder, and should be decided according to the judgment of the practitioner and each patient's circumstances. However, suitable dosage ranges for oral administration are generally about 0.001 milligram to about 200 milligrams of (+)-1-(3,4-dichlorophenyl)-3-azabicyclo[3.1.0]hexane or a pharmaceutically acceptable salt thereof, per kilogram body weight, per day. In various embodiments, oral dosage amounts are between about 0.01 milligram to about 100 milligrams per kilogram body weight per day, between about 0.1 milligram to about 75 milligrams per kilogram body weight per day, between about 0.5 milligram to about 50 milligrams per kilogram body weight per day, or between about 1 to 40 milligrams per kilogram body weight per day, and in certain embodiments between about 1 milligram to 30 milligrams, or between about 1 milligram to 3 milligrams per kilogram body weight per day. The dosage amounts described herein refer to total amounts administered; that is, if (+)-1-(3,4-dichlorophenyl)-3-azabicyclo[3.1.0]hexane and/or one or more pharmaceutically acceptable salts thereof are administered, the specified dosages correspond to the total amount administered. Oral compositions will typically contain about 10% to about 95% of the active ingredient by weight.

Exemplary dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of (+)-1-(3,4-dichlorophenyl)-3-azabicyclo[3.1.0]hexane or a pharmaceutically acceptable salt thereof per kilogram body weight per day and comprise active ingredient in the range of about 0.5% to about 10% by weight.

Exemplary dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

Pharmaceutical formulations of a (+)-1-(3,4-dichlorophenyl)-3-azabicyclo[3.1.0]hexane compound of the invention may include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives. The compositions of the invention for controlling appetite and/or treating weight gain and obesity and associated conditions and complications can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent. If desired, the (+)-1-(3,4-dichlorophenyl)-3-azabicyclo[3.1.0]hexane compound of the invention can be administered in a controlled release form by use of a slow release carrier, such as a hydrophilic, slow release polymer. In certain embodiments, (+)-1-(3,4-dichlorophenyl)-3-azabicyclo[3.1.0]hexane compositions may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

(+)-1-(3,4-dichlorophenyl)-3-azabicyclo[3.1.0]hexane compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, and other dosage forms contemplated herein, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds.

Additional compositions and methods of the invention are provided for topical administration of (+)-1-(3,4-dichlorophenyl)-3-azabicyclo[3.1.0]hexane compositions for the treatment of obesity. Topical compositions may comprise (+)-1-(3,4-dichlorophenyl)-3-azabicyclo[3.1.0]hexane compositions and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. Yet additional (+)-1-(3,4-dichlorophenyl)-3-azabicyclo[3.1.0]hexane formulations are provided for parenteral administration, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. The formulations and ingredients will typically be sterile or readily sterilizable, biologically inert, and easily administered.

As noted above, in certain embodiments the methods and compositions of the invention may employ pharmaceutically acceptable salts, e.g., acid addition or base salts of the above-described (+)-1-(3,4-dichlorophenyl)-3-azabicyclo[3.1.0]hexane compounds and/or related or derivative compounds. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts may also be formed. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like. Suitable base salts are formed from bases that form non-toxic salts, for example aluminum, calcium, lithium, magnesium, potassium, sodium, zinc and diethanolamine salts, sulfate, citrate, acetate, oxalate, chloride, bromide, iodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

In other detailed embodiments, the methods and compositions of the invention employ prodrugs of (+)-1-(3,4-dichlorophenyl)-3-azabicyclo[3.1.0]hexane. Prodrugs are considered to be any covalently bonded carrier which releases the active parent drug in vivo. Examples of prodrugs useful within the invention include esters or amides with hydroxyalkyl or aminoalkyl as a substituent, and these may be prepared by reacting such compounds as described above with anhydrides such as succinic anhydride.

The invention disclosed herein will also be understood to encompass methods and compositions comprising a compound or derivative compound of Formula I using in vivo metabolic products of the said compounds (either generated in vivo after administration of the subject precursor compound, or directly administered in the form of the metabolic product itself). Such products may result, for example, from the oxidation, reduction, hydrolysis, amidation, esterification and the like of the administered compound, primarily due to enzymatic processes. Accordingly, the invention includes methods and compositions of the invention employing compounds produced by a process comprising contacting a compound or derivative compound of Formula I with a mammalian subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The above disclosure generally describes the present invention. A more complete understanding can be obtained by referring to the following examples. These examples are described solely for purposes of illustration and are not intended to limit the scope of the invention. Although specific terms have been employed herein, such terms are intended for descriptive use and not for purposes of limitation.

### EXAMPLES

Utilizing in vivo analytical methods, it is demonstrated herein that (+)-1-(3,4-dichlorophenyl)-3-azabicyclo[3.1.0]hexane possesses appetite suppressant activity. This novel use may be related to (+)-1-(3,4-dichlorophenyl)-3-azabicyclo[3.1.0]hexane's ability to modulate serotonin and norepinephrine uptake. Insights into the possible mechanism by which (+)-1-(3,4-dichlorophenyl)-3-azabicyclo[3.1.0]hexane demonstrates its obesity treating activity was provided by transporter assays for norepinephrine and serotonin. (+)-1-(3,4-dichlorophenyl)-3-azabicyclo[3.1.0]hexane has a significantly greater affinity for norepinephrine and serotonin than (±)-1-(3,4-dichlorophenyl)-3-azabicyclo[3.1.0]hexane indicating potentially greater activity and effectiveness than a racemic mixture.

### Example 1

### Resolution of (+)-1-(3,4-dichlorophenyl)-3-azabicyclo[3.1.0]hexane via chiral chromatography

To 279 mg of (±)-1-(3,4-dichlorophenyl)-3-azabicyclo[3.1.0]hexane hydrochloride obtained using the methods described in Epstein et al., J. Med. Chem., 24:481-490 (1981) was added 7 mL of 9:1 hexane:isopropyl alcohol, followed by 8 drops of diethylamine. To the resulting mixture was added isopropyl alcohol, dropwise, until a solution was obtained. The solution was concentrated to a volume of 6 mL using a stream of helium gas. Six 1-mL portions of the concentrate were subjected to high-performance liquid chromatography using an HPLC instrument equipped with a 1 cm×25 cm Daicel CHIRALPAK AD column (Chiral Technologies, Inc., Exton, Pa.). Elution was carried out at ambient temperature using 95:5 (v/v) hexane:isopropyl alcohol solution containing 0.05% diethylamine as a mobile phase at a flow rate of 6 mL/min. The fraction eluting at about 21.5 to 26 minutes was collected and concentrated to provide a first residue, which was dissolved in a minimal amount of ethyl acetate. Using a stream of nitrogen, the ethyl acetate solution was evaporated to provide a second residue, which was dissolved in 1 mL of diethyl ether. To the diethyl ether solution was added 1 mL diethyl ether saturated with gaseous hydrochloric acid. A colorless precipitate formed, was filtered, washed with 2 mL of diethyl ether and dried to provide 73.4 mg of (+)-1-(3,4-dichlorophenyl)-3-azabicyclo[3.1.0]hexane hydrochloride: optical rotation [α]²⁵_{D}=+60° in methanol at 2 mg/mL; 99.7% enantiomeric excess.

### Example 2

### Resolution of (+)-1-(3,4-dichlorophenyl)-3-azabicyclo[3.1.0]hexane the use of 1-di-(o-benzoyl) tartaric acid as a chiral resolving agent

A 2.68 g (0.0101 mol) sample of (±)-1-(3,4-dichlorophenyl)-3-aza-bicyclo[3.1.0]hexane hydrochloride as described in Epstein, et al., J. Med. Chem., 1981, 24, pp. 481-490, was dissolved in 50 mL of water and this solution was made basic to pH 11 with 10N sodium hydroxide solution, and the precipitated free base was extracted into 25 mL of dichloromethane. This solution was dried over sodium sulfate and filtered. To this filtrate, was added a solution of 3.70 g (0.1030 mol) of L-di-(O-benzoyl)tartaric acid in 25 mL of methanol, and this solution was boiled until crystallization ensued. The mixture was cooled to room temperature and allowed to stand for one hour. The crystals were collected to give 3.21 g of colorless crystals which were boiled in 50 mL of methanol, and this mixture was cooled in an ice bath, then filtered to give 2.04 g of colorless crystals, m.p. 185-187° C. of (+)-1-(3,4-dichlorophenyl)-3-azabicyclo[3.1.0]hexane monosalt with L-di-(O-benzoyl) tartaric acid. This salt was stirred with 5N aqueous sodium hydroxide and the liberated free base was extracted into ethyl acetate. The organic layer was washed with dilute aqueous sodium hydroxide solution, then water, and then dried over sodium sulfate. This was filtered, and the filtrate was treated with a solution of HCl in ether until precipitation ceased. The crystals were collected by filtration and air dried to yield 0.748 g of (+)-1-(3,4-dichlorophenyl)-3-azabicyclo[3.1.0]hexane hydrochloride as colorless crystals, m.p. 173-173° C., (α)=+64.2°, C.=6.7, MeOH, which was substantially free of the corresponding (−)-enantiomer.

### Example 3

### Comparison of activity of (+)-1-(3,4-dichlorophenyl)-3-azabicyclo[3.1.0]hexane in Norepinephrine, and Serotonin Transporter Binding Assays

Norepinephrine and serotonin uptake inhibition activity of (+)-1-(3,4-dichlorophenyl)-3-azabicyclo[3.1.0]hexane HCL was compared to that of (±)-1-(3,4-dichlorophenyl)-3-azabicyclo[3.1.0]hexane HCL using standard transporter binding assays.

### Norepinephrine Transporter Binding Assay

The norepinephrine transporter binding assay was performed according to the methods described in Raisman et al., 1982, Eur. Jrnl. Pharmacol. 78:345-351 and Langer et al., 1981, Eur. Jrnl. Pharmacol. 72:423. The receptor source was rat forebrain membranes; the radioligand was [₃H]nisoxetine (60-85 Ci/mmol) at a final ligand concentration of 1.0 nM; the non-specific determinant [1.0 μm]; reference compound and positive control were (±)-desmethylimipramine HCl. (+)-1-(3,4-dichlorophenyl)-3-azabicyclo[3.1.0]hexane HCl was obtained as described above. Reactions were carried out in 50 mM TRIS-HCl (pH 7.4), containing 300 mM NaCl and 5 mM KCl at 0° C. to 4° C. for 4 hours. The reaction was terminated by rapid vacuum filtration onto glass fiber filters. Radioactivity trapped in the filters was determined and compared to control values in order to ascertain the interactions of the test compound with the norepinephrine uptake site. The data are reported in Table 1 below.

TABLE 1
Norepinephrine Transporter Binding Assay

| Compound | Ki (M) |
|---|---|
| (±)-1-(3,4-dichlorophenyl)-3-azabicyclo[3.1.0]hexane HCl | 1.42 × 10⁻⁷ |
| (+)-1-(3,4-dichlorophenyl)-3-azabicyclo[3.1.0]hexane HCl | 8.20 × 10⁻⁸ |
| (±) desmethylimprimine HCL | 1.13 × 10⁻⁹ |

### Serotonin Transporter Binding Assay

The serotonin transporter binding assay was performed according to the methods described in D'Amato et al., 1987, Jrnl. Pharmacol. & Exp. Ther. 242:364-371 and Brown et al., 1986, Eur. Jrnl. Pharmacol. 123:161-165. The receptor source was rat forebrain membrane; the radioligand was [³H]citalopram (70-87 Ci/mmol) at a final ligand concentration of 0.7 nM; the non-specific determinant was 10 μM clomipramine, a high-affinity serotonin uptake inhibitor. The reference compound and positive control were (±)-desmethylimipramine. The test compound, (+)-1-(3,4-Dichlorophenyl)-3-azabicyclo[3.1.0]hexane HCl was obtained according to the methods above. Reactions were carried out in 50 mM TRIS-HCl (pH 7.4) containing 120 mM NaCl and 5 mM KCl at 25° C. for 60 minutes. The reaction was terminated by rapid vacuum filtration onto glass fiber filters. Radioactivity trapped in the filters was determined using liquid scintillation spectrometry and compared to control values in order to ascertain any interactions of test compound with the serotonin transporter binding site. The data are reported in Table 2 below.

TABLE 2
Serotonin Transporter Binding Assay

| Compound | Ki |
|---|---|
| (±)-1-(3,4-dichlorophenyl)-3-azabicyclo[3.1.0]hexane HCl | 1.18 × 10⁻⁷ |
| (+)-1-(3,4-dichlorophenyl)-3-azabicyclo[3.1.0]hexane HCl | 5.08 × 10⁻⁸ |
| (±) desmethylimiprimine HCL | 2.64 × 10⁻⁸ |

The data in Tables 1 and 2 show that (+)-1-(3,4-dichlorophenyl)-3-azabicyclo[3.1.0]hexane HCl has a significantly greater affinity for the norepinephrine uptake site and the serotonin uptake site than does the (±)-1-(3,4-dichlorophenyl)-3-azabicyclo[3.1.0]hexane HCl or controls. Therefore, (+)-1-(3,4-dichlorophenyl)-3-azabicyclo[3.1.0]hexane or a pharmaceutically acceptable salt thereof will be significantly more active than (±)-1-(3,4-dichlorophenyl)-3-azabicyclo[3.1.0]hexane or a pharmaceutically acceptable salt thereof.

### Example 4

### Comparison of activity of (+)-1-(3,4-dichlorophenyl)-3-azabicyclo[3.1.0]hexane in Norepinephrine (NE), Dopamine (DA) and Serotonin (5-HT) Human Transporter Binding Assays

Human embryonic kidney (HEK-293) cells stably transfected and constitutively expressing the human norepinephrine transporter (hNET; Pacholezyk et al., Nature, 350:350-354 (1991)), the human dopamine transporter (hDAT; Pristupa et al., Mol. Pharmacol., 45:125-135 (1994)), or the human serotonin transporter (hSERT; Ramamoorthy et al., Proc. Natl. Acad. Sci. U.S.A. 90:2542-2546 (1993)) were grown and passaged in 150-mm petri dishes with 17.5 ml of Dulbecco's modified Eagle's medium (MEM; Mediatech Inc., Herndon, Va.) containing 0.1 mM non-essential amino acid solution for MEM (Mediatech Inc.), 5% (v/v) fetal clone bovine serum product (Hyclone Laboratories, Logan, Utah), and 1 U/μL penicillin/streptomycin solution (Mediatech, Inc.). The cells were incubated in 10% CO₂, 90% air at 37° C. and 100% humidity. The hNET cell cultures contained 250 μg/mL geneticin sulfate. The cells were grown to 70-80% confluency prior to harvesting.

Cell membranes containing hSERT, hNET, or hDAT were prepared from the cell lines to assay ligand binding for each of the transporters. Briefly, the cell medium was removed by aspiration, and the cells were washed with 4 mL modified Puck's D1 solution (solution 1; Richelson et al. in “Methods in Neurotransmitter Receptor Analysis” Yamamura, H. I.; Enna, S. J.; Kuhar, M. J. Eds.; New York, Raven Press, 1990, pp 147-175). The washed cells were incubated for 5 minutes at 37° C. in 10 mL solution 1 containing 100 mM ethylene glycol-bis N,N,N′,N′-tetraacetic acid (EGTA). The cells were then scraped from the flask surface with a rubber spatula, placed into a centrifuge tube, and collected by centrifugation at 1000×g for 5 minutes at 4° C. The resulting supernatant was discarded, and the cell pellet was resuspended in 0.5 to 1.0 mL of the appropriate binding buffer (described below). The resuspended cell pellet was homogenized using a Polytron for 10 seconds at setting 6. The resulting homogenate was centrifuged at about 36,000×g for 10 minutes at 4° C. The supernatant was discarded and the pellet was resuspended in the same volume of the appropriate binding buffer and centrifuged again. The supernatant was discarded and the final pellet containing cell membranes was resuspended in the appropriate binding buffer and stored at −80° C. until use. The final protein concentration was determined by the Lowry assay using bovine serum albumin as a standard (Lowry et al., J. Biol. Chem. 193:265-275 (1951)).

Radioligand binding assays for the indicated transporters were performed as follows. To assess binding to the cloned hSERT, cells expressing hSERT were homogenized in 50 mM Tris-HCl with 120 mM NaCl and 5 mM KCl (pH 7.4). The binding reaction consisted of 30 μg cell membrane protein, 1.0 nM [³H]imipramine (imipramine hydrochloride, benzene ring-³H, specific activity 46.5 Ci/mmol; Dupont New England Nuclear, Boston, Mass.), and varying concentrations of either unlabeled imipramine or the test compound. A reaction to determine non-specific binding consisted of 15 μg cell membrane protein, 1.0 nM [³H]imipramine, and 1 μM final concentration of unlabeled imipramine. The reactions were incubated at 22° C. for 60 minutes. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman Instruments, Fullerton, Calif.). Radioactivity was measured with a Beckman liquid scintillation counter (LS 5000TD).

To assess binding to the cloned hNET, cells expressing hNET were homogenized in 50 mM Tris-HCl with 300 mM NaCl and 5 mM KCl (pH 7.4). The binding reaction consisted of 25 μg cell membrane protein, 0.5 nM [³H]nisoxetine (nisoxetine HCl, [N-methyl-3H], specific activity 85.0 Ci/mmol; Amersham, Arlington Hts., Ill.), and varying concentrations of either unlabeled nisoxetine or the test compound. A reaction to determine non-specific binding consisted of 25 μg cell membrane protein, 0.5 nM [³H]nisoxetine, and 1 μM final concentration of unlabeled nisoxetine. The reactions were incubated at 22° C. for 60 minutes. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman Instruments, Fullerton, Calif.). Radioactivity was measured with a Beckman liquid scintillation counter (LS 5000TD).

To assess binding to the cloned hDAT, cells expressing hDAT were homogenized in 50 mM Tris-HCl with 120 mM NaCl (pH 7.4). The binding reaction contained 30 μg cell membrane protein, 1 nM [³H]WIN35428 (WIN35428, [N-methyl-³H], specific activity 83.5 Ci/mmol; Dupont New England Nuclear, Boston, Mass.), and varying concentrations of either unlabeled WIN35428 or the test compound. A reaction to determine non-specific binding contained 30 μg cell membrane protein, 1 nM [³H]WIN35428, and 10 μM final concentration of unlabeled WIN35428. The reactions were incubated at 22° C. for 1 hour. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman Instruments, Fullerton, Calif.). Radioactivity was measured with a Beckman liquid scintillation counter (LS 5000TD).

TABLE 3

| | hDAT, | hSERT, | hNET, |
|---|---|---|---|
| Compound | Binding | Binding | Binding |
| (+)-1-(3,4-dichlorophenyl)-3- | 213 ± 56 | 99 ± 16 | 262 ± 41 |
| azabicyclo[3.1.0]hexane | | | |
| (±)-1-(3,4-dichlorophenyl)-3- | 186 ± 40 | 188 ± 28 | 378 ± 43 |
| azabicyclo[3.1.0]hexane | | | |
| Imipramine | | 1.7 | |
| WIN35428 | 29 | | |
| Nisoxetine | | | 2.4 |

### Example 5

### Comparison of potency of (+)-1-(3,4-dichlorophenyl)-3-azabicyclo[3.1.0]hexane in blocking [³H]DA, [³H]5-HT, or [³H]NE uptake by cell lines expressing recombinant human DA, 5-HT or NE transporters

The potency of (+)-1-(3,4-dichlorophenyl)-3-azabicyclo[3.1.0]hexane (DOV 21947) in suppressing monoamine neurotransmitter uptake was determined using suspensions of cell lines recombinantly expressing human transporters. These suspensions were prepared by removing the medium from cells grown on 150 mm diameter tissue culture dishes, then washing the plates twice with Ca²⁺, Mg²⁺-free phosphate buffered saline. Fresh Ca²⁺, Mg²⁺-free PBS (2.5 mL) was then added to each plate and the plates placed into a 25° C. water bath for 5 min. The cells were gently scraped from the plates and cell clusters separated by trituration with a pipette for 5-10 aspiration/ejection cycles (Eshleman, et al., J. Pharmacol Exp Ther 289: 877-885, 1999). To these suspensions were added bicifadine, Krebs-HEPES assay buffer, and, after a 10 minute pre-incubation of the isolated cells at 25° C., either [³H]DA, [³H]5-HT, or [³H]NE, (56, 26.9, 60 Ci/mmol, respectively, 20 nM final concentration). The assay was incubated an additional 10 minutes, and the radiolabelled neurotransmitter uptake terminated by vacuum filtration. Specific uptake was defined as the difference in uptake observed in the absence and presence of 5 μM mazindol (hDAT and hNET) or 5 μM imipramine (hSERT).

Table 4 outlines the results of the monoamine neurotransmitter uptake study. (+)-1-(3,4-dichlorophenyl)-3-azabicyclo[3.1.0]hexane (DOV 21947) potently inhibited the uptake of all three monoamine neurotransmitters tested. In contrast, the reference agents, which are clinically available treatments for depression, preferentially inhibited the uptake of 5-HT and NE, and showed low or no potency in blocking DA uptake.

TABLE 4
(+)-1-(3,4-dichlorophenyl)-3-azabicyclo[3.1.0]hexane
blocks the uptake of 5-HT, NE and DA by cell lines recombinantly
expressing human 5-HT, NE and DA transporters.

| Drug | 5-HT Uptake | NE Uptake | DA Uptake |
|---|---|---|---|
| DOV 21,947¹ | 11.3 | 20.9 | 94 |
| Duloxetine² | 3.7 | 20 | 439 |
| Venlafaxine² | 145 | 1420 | 3070 |
| Milnacipran² | 151 | 68 | >1000000 |

Values are expressed in nM; ¹as IC₅₀ or²Kᵢ. Duloxetine, venlafaxine and milnacipran data are taken from Vaishnavi et al., Biol. Psychiatry, 55: 320-322, 2004.

### Example 5

### Measurement of the effect of (+)-1-(3,4-dichlorophenyl)-3-azabicyclo[3.1.0]hexane on weight gain and food intake

Male Sprague-Dawley rats (Charles River Laboratories) were used to establish the diet-induced models of obesity (DIO). The animals were housed and fed in facilities maintained on a standard 12-h light/dark cycle (lights on 6:00 AM; lights off, 6:00 PM) at a room temperature of 19.5-24.5° C. and relative humidity of 45-65%. All animals had free access to water. At four weeks of age, the rats were made obese by switching to the moderately high fat diet (Research Diets; D122668B) in pellet form. The rats were housed in a group environment until one week before the study (body weight approximately 550-625 g) when they were singly housed in cages with an automated food intake monitoring system (AFIS), where consumption of a milled pellet form of the same diet was measured for the duration of the studies. Test compounds or vehicle were then orally administered in a volume of 5 mL/kg 60 minutes before access to food and water (0900 hrs).

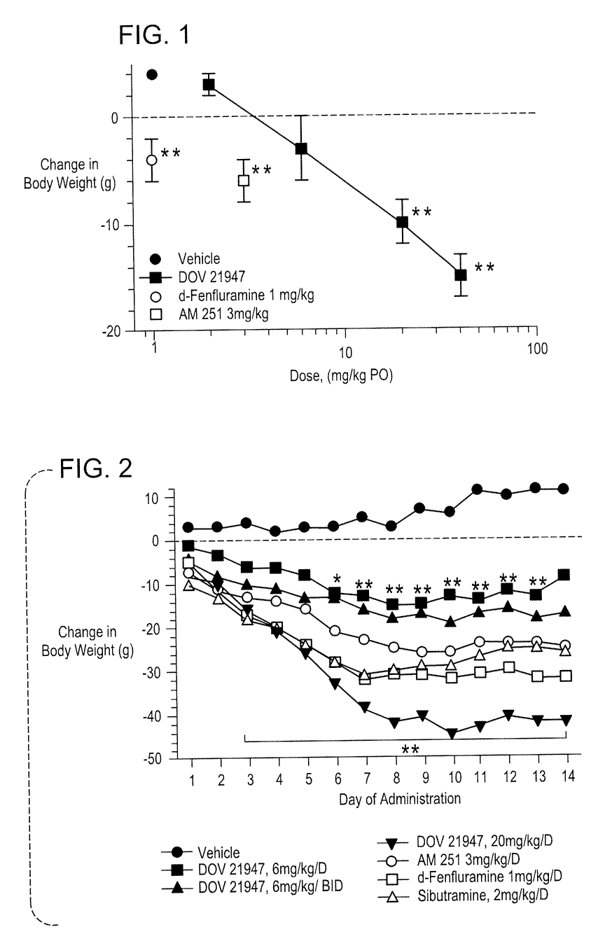

Body weight was measured prior to and 18 hours after administration of the various compounds. As can be seen in FIG. 1, (+)-1-(3,4-dichlorophenyl)-3-azabicyclo[3.1.0]hexane (DOV 21947) dose-dependently suppressed this body weight gain compared to vehicle treated animals. Furthermore, doses at 20 and 40 mg/kg of (+)-1-(3,4-dichlorophenyl)-3-azabicyclo[3.1.0]hexane was significantly more effective than either AM251 or d-FEN1.

Each point in FIG. 1 represents the MEAN ±SEM of results from 5-10 animals. *: Significantly different from contemporaneous vehicle control, P<0.05, 2-way ANOVA followed by Bonferroni's test.

The ability of (+)-1-(3,4-dichlorophenyl)-3-azabicyclo[3.1.0]hexane (DOV 21947) to sustain the decrease in weight gain was studied using a 14 day administration protocol. The results are shown in FIG. 2. Daily administration of DOV 21947 (6 mg/kg/D PO) significantly reduced body weight from days 6-13. Increasing the dose of DOV 21947 (6 mg/kg/BID, 20 mg/kg/D, PO) significantly reduced body weight as early as 3 days after administration, an effect maintained over the remainder of the study. AM 251, dexfenfluramine and sibutramine had a similar effect.

Each point in FIG. 2 represents the MEAN ±SEM of results from 7 animals. **: Significantly different from vehicle over the range of days indicated for DOV 21947 (6 mg/kg, 6 mg/kg BID, 20 mg/kg/D), AM251, dexfenfluramine or sibutramine treatment groups, 2-way ANOVA, Bonferroni adjusted post-hoc test.

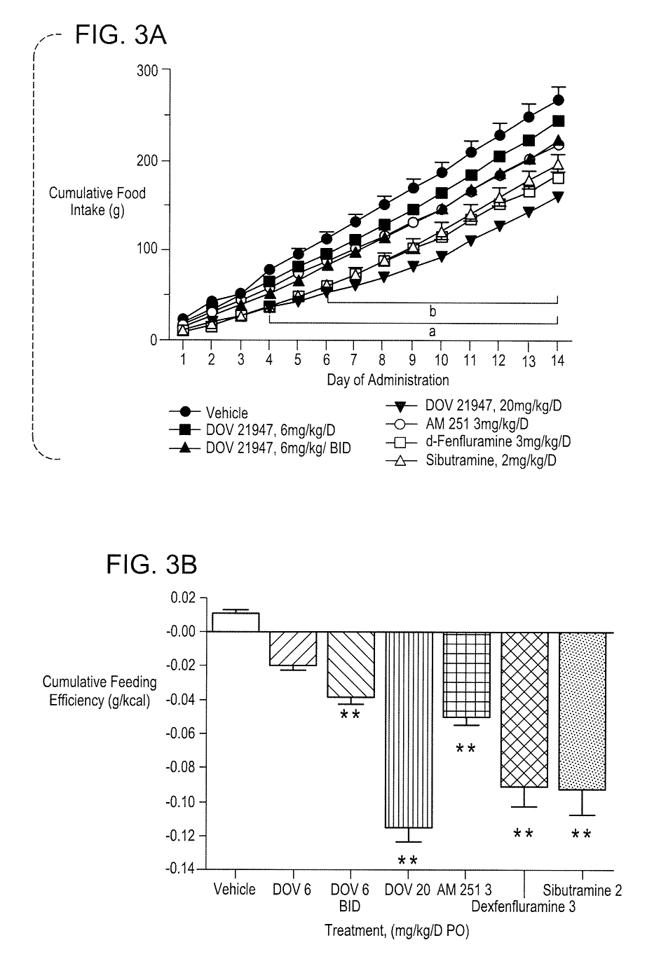

The effects of (+)-1-(3,4-dichlorophenyl)-3-azabicyclo[3.1.0]hexane (DOV 21947) on cumulative food intake and feeding efficiency in DIO rats over 14 days of daily administration is shown in FIG. 3. (+)-1-(3,4-dichlorophenyl)-3-azabicyclo[3.1.0]hexane (DOV 21947, 6 mg/kg BID, 20 mg/kg/D) significantly decreased cumulative food intake by DIO rats from days 4-14 of testing (Panel A). Cumulative feeding efficiency was also reduced by treatment with (+)-1-(3,4-dichlorophenyl)-3-azabicyclo[3.1.0]hexane (DOV 21947) and reference standards (Panel B). Feeding efficiency was determined as the Change in body weight (g)/Feeding efficiency (kcal) over Days 0-14 of the study.

Panel A. a: Significantly different from vehicle-treated rat values, DOV 21947 (6 mg/kg BID, 20 mg/kg/D), dexfenfluramine and sibutramine (days 4-14). b: Significantly different from vehicle-treated rat values, AM251 (days 6-14). Panel B **: Significantly different from vehicle-treated rat values, P<0.01, 1-way ANOVA followed by Dunnet's post-hoc comparison test.

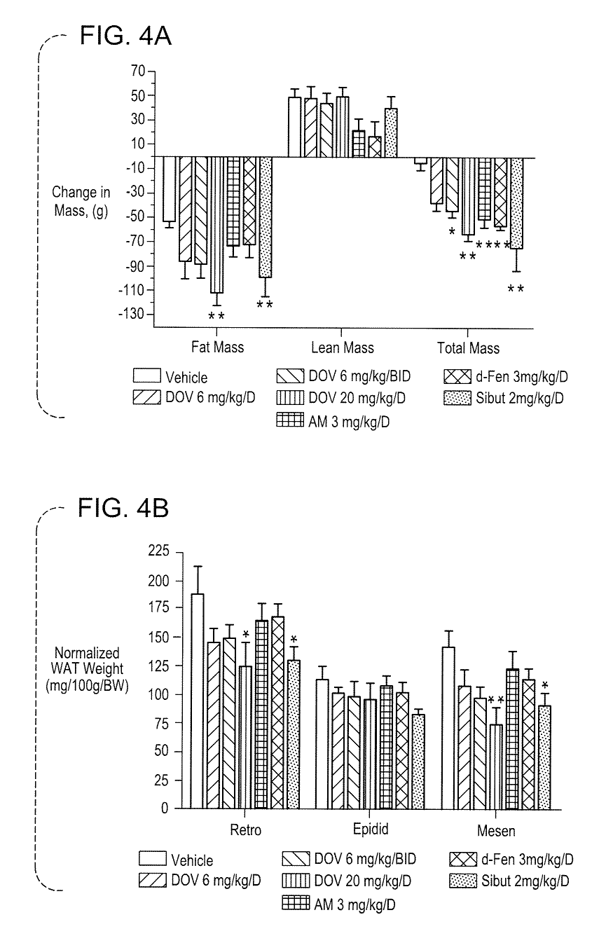

The decrease in body weight induced by 14 days of administration of (+)-1-(3,4-dichlorophenyl)-3-azabicyclo[3.1.0]hexane (DOV 21947) resulted from a selective decrease in fat mass, as depicted in FIG. 4. Panel A. DOV 21947 (6 mg/kg BID, 20 mg/kg/D), AM 251, dexfenfluramine, and sibutramine significantly reduced total body mass. This was due to a selective loss of fat mass following DOV 21947 (20 mg/kg/D) and sibutramine treatment. Panel B. The measurement of the distribution of WAT indicated that DOV 21947 (20 mg/kg/D) and sibutramine selectively reduced the mass of retroperitoneal and mesenteric, but not epididymal fat deposits. Regional fat masses determined following manual dissection, and normalized to total body mass.

Each bar represents the MEAN ±SEM of observations from 7 animals. *, **: Significantly different from vehicle treated animal values, P<0.05, 0.01, respectively, 1-way ANOVA, Dunnet's post-hoc test.

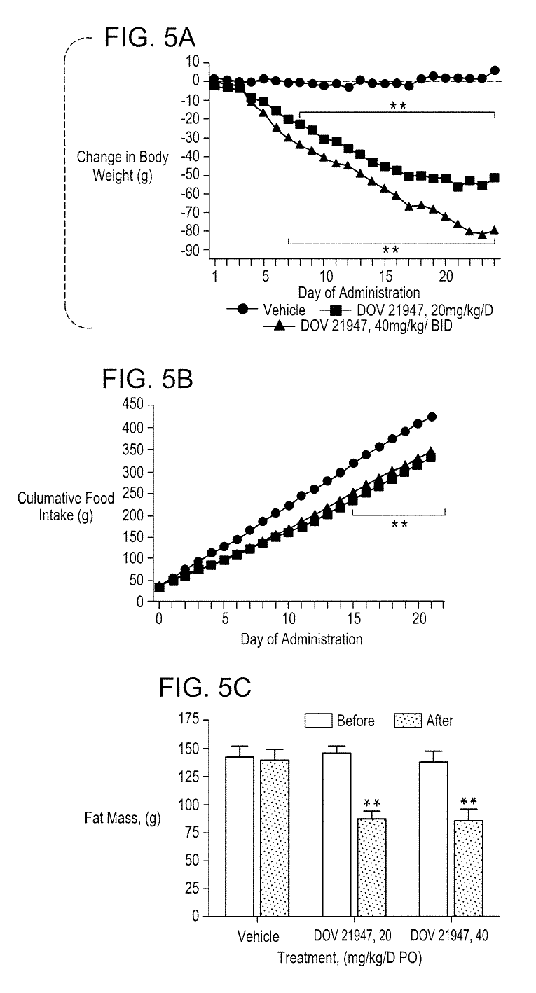

The effect of (+)-1-(3,4-dichlorophenyl)-3-azabicyclo[3.1.0]hexane (DOV 21947) on body weight, food intake and body composition in DIO rats after 21 to 24 days of administration (FIG. 5). Panel A. Both doses of DOV 21947 (20, 40 mg/kg/D) significantly decreased the total body weight (g) of DIO rats from days 10 to 24 (20 mg/kg/D) and 7 to 24 (40 mg/kg/D) of administration. Panel B. DOV 21947 induced a significant decrease in cumulative food intake manifested 15-21 days into the administration period. Panel C. Both doses of DOV 21947 (20, 40 mg/kg/D) significantly decreased fat mass after 21 days of administration.

Each point represents the MEAN, and each bar the MEAN ±SEM of observations from 10 rats/group. **: Significantly different from vehicle levels for both DOV 21947 dosage groups, P<0.01, 2-way ANOVA, Bonferroni adjusted post-hoc analysis.

FIG. 6 depicts the decrease in plasma triglyceride levels after 14 days of administration of (+)-1-(3,4-dichlorophenyl)-3-azabicyclo[3.1.0]hexane (DOV 21947). Only DOV 21947 (6 mg/kg BID, 20 mg/kg/D) and sibutramine significantly decreased plasma triglyceride levels after 14 days of administration.

Each bar represents the MEAN ±SEM of observations from 7 rats/group *, **: Significantly different from vehicle treated group, P<0.05, 0.01, respectively, 1-way ANOVA and Dunnett's test.

### Example 6

### Toxicity Studies in Rats for (+)-1-(3,4-Dichlorophenyl)-3-azabicyclo[3.1.0]hexane hydrochloride

One hundred and forty Crl:CD®(SD)IGS BR rats were divided into 7 groups of 20 rats (ten male and ten female). The selected animals were approximately seven to eight weeks old at the initiation of dose administration; body weight values ranged from 203 g to 250 g for males and from 154 g to 192 g for females in the toxicology groups and from 207 g to 247 g for males and from 157 g to 197 g for females in the toxicokinetic groups. Individual body weights were recorded at least weekly, beginning approximately two weeks prior to test article administration (study week −2). Mean body weights and mean body weight changes were calculated for the corresponding intervals. Final body weights (fasted) were recorded prior to the scheduled necropsy.

The test rats in groups 2-4 and 2A-4A were given 10, 25 and 60 mg/kg/day (+)-1-(3,4-dichlorophenyl)-3-azabicyclo[3.1.0]hexane hydrochloride respectively in deionized water orally once daily for a minimum of 91 consecutive days. A concurrent toxicology control group (Group 1) received the vehicle on a comparable regimen.

For toxicology assessment, all animals were observed three times daily for mortality and moribundity. Clinical examinations were performed daily and detailed physical examinations were performed weekly. Following 13 weeks of dose administration, all surviving animals were euthanized. Complete necropsies were conducted on all animals, selected organs were weighed and selected tissues were examined microscopically from all animals.

For toxicokinetic evaluation, all animals were observed twice daily for mortality and moribundity. Blood samples were collected from three animals/sex/group at 0 (pre-dose), 1, 2, 4, 8 and 24 hours after dose administration on study days 0 and 87. All toxicokinetic animals were euthanized and discarded following the final blood collection (study day 88).

Body weight gains in the groups receiving 25 and 60 mg/kg/day were lower throughout the study. (FIGS. 2 and 3) By the end of the study, mean cumulative body weight gains were 30% and 13% lower than the control in the 60 mg/kg/day group males and females, respectively, and 16% and 13% lower than control in the 25 mg/kg/day group males and females, respectively (FIGS. 4 and 5). Mean body weights were 18% and 9% lower than control in 60 mg/kg/day males and females respectively, and 10% lower than control in 25 mg/kg/day males by the end of the study (FIGS. 2 and 3). The lower body weight gains were accompanied by lower food consumption during the first two weeks of treatment in the 60 mg/kg/day group (19% and 32% lower for males and females, respectively, during the first week and 4% and 11% lower for males and females, respectively, during the second week) and during the first week of treatment in the 25 mg/kg/day group (8% and 16% lower for males and females, respectively).

Mean consumption was significantly (p<0.01) lower in the 60 mg/kg/day group males and females and 25 mg/kg/day group females during study week 0 to 1 when compared to the control group (Table 5). Mean food consumption was also significantly (p<0.05) lower in the 60 mg/kg/day group females during study week 1 to 2 (Table 5). There were no other remarkable changes in food consumption.

TABLE 5
SUMMARY OF WEEKLY FOOD CONSUMPTION - (C/ANIMAL/DAY)

| | | ----- F E M A L E ----- | | | |
|---|---|---|---|---|---|
| | Group: | 0 MG/KG/DAY | 10 MG/KG/DAY | 25 MG/KG/DAY | 60 MG/KG/DAY |
| WEEK | −2 TO −1 | | | | |
| | MEAN | 18. | 18. | 17. | 17. |
| | S.D. | 1.2 | 0.8 | 1.3 | 1.2 |
| | N | 10 | 10 | 10 | 10 |
| | 0 TO 1 | | | | |
| | MEAN | 19. | 17. | 16.** | 13.** |
| | S.D. | 1.3 | 1.4 | 1.1 | 1.9 |
| | N | 10 | 10 | 10 | 6 |
| | 1 TO 2 | | | | |
| | MEAN | 19. | 19. | 20. | 17.* |
| | S.D. | 1.1 | 1.7 | 1.8 | 1.8 |
| | N | 10 | 10 | 10 | 6 |
| | 2 TO 3 | | | | |
| | MEAN | 20. | 20. | 19. | 19. |
| | S.D. | 2.2 | 2.0 | 1.5 | 2.4 |
| | N | 10 | 10 | 10 | 6 |
| | 3 TO 4 | | | | |
| | MEAN | 20. | 20. | 20. | 18. |
| | S.D. | 1.5 | 2.6 | 2.7 | 0.6 |
| | N | 10 | 10 | 10 | 6 |
| | 4 TO 5 | | | | |
| | MEAN | 19. | 19. | 18. | 17. |
| | S.D. | 1.8 | 1.9 | 1.0 | 1.2 |
| | N | 10 | 10 | 10 | 6 |
| | 5 TO 6 | | | | |
| | MEAN | 18. | 18. | 18. | 18. |
| | S.D. | 2.0 | 1.7 | 1.7 | 1.5 |
| | N | 10 | 10 | 10 | 6 |
| | 6 TO 7 | | | | |
| | MEAN | 18. | 18. | 18. | 18. |
| | S.D. | 1.9 | 2.2 | 1.9 | 1.1 |
| | N | 10 | 10 | 10 | 6 |
| | 7 TO 8 | | | | |
| | MEAN | 17. | 18. | 17. | 18. |
| | S.D. | 1.9 | 2.8 | 1.6 | 1.6 |
| | N | 10 | 10 | 10 | 6 |
| | 8 TO 9 | | | | |
| | MEAN | 18. | 18. | 19. | 18. |
| | S.D. | 2.3 | 2.6 | 2.0 | 1.2 |
| | N | 10 | 10 | 10 | 6 |
| WEEK | 9 TO 10 | | | | |
| | MEAN | 17. | 18. | 18. | 17. |
| | S.D. | 3.2 | 1.8 | 1.5 | 1.2 |
| | N | 10 | 10 | 10 | 6 |
| | 10 TO 11 | | | | |
| | MEAN | 17. | 18. | 18. | 17. |
| | S.D. | 2.6 | 1.9 | 1.6 | 2.3 |
| | N | 10 | 10 | 10 | 6 |
| | 11 TO 12 | | | | |
| | MEAN | 18. | 18. | 18. | 18. |
| | S.D. | 1.8 | 1.6 | 1.3 | 1.3 |
| | N | 10 | 10 | 10 | 6 |
| | 12 TO 13 | | | | |
| | MEAN | 18. | 17. | 17. | 17. |
| | S.D. | 2.5 | 2.3 | 3.7 | 1.0 |
| | N | 10 | 10 | 10 | 6 |
| WEEK | −2 TO −1 | | | | |
| | MEAN | 18. | 18. | 17. | 17. |
| | S.D. | 1.2 | 0.8 | 1.3 | 1.2 |
| | N | 10 | 10 | 10 | 10 |
| | 0 TO 1 | | | | |
| | MEAN | 19. | 17. | 16.** | 13.** |
| | S.D. | 1.3 | 1.4 | 1.1 | 1.9 |
| | N | 10 | 10 | 10 | 6 |
| | 1 TO 2 | | | | |
| | MEAN | 19. | 19. | 20. | 17.* |
| | S.D. | 1.1 | 1.7 | 1.8 | 1.8 |
| | N | 10 | 10 | 10 | 6 |
| | 2 TO 3 | | | | |
| | MEAN | 20. | 20. | 19. | 19. |
| | S.D. | 2.2 | 2.0 | 1.5 | 2.4 |
| | N | 10 | 10 | 10 | 6 |
| | 3 TO 4 | | | | |
| | MEAN | 20. | 20. | 20. | 18. |
| | S.D. | 1.5 | 2.6 | 2.7 | 0.6 |
| | N | 20 | 10 | 10 | 6 |
| | 4 TO 5 | | | | |
| | MEAN | 19. | 19. | 18. | 17. |
| | S.D. | 1.8 | 1.9 | 1.9 | 1.2 |
| | N | 20 | 10 | 10 | 6 |
| | 5 TO 6 | | | | |
| | MEAN | 19. | 18. | 19. | 19. |
| | S.D. | 2.0 | 1.7 | 1.7 | 1.5 |
| | N | 10 | 10 | 10 | 6 |
| | 6 TO 7 | | | | |
| | MEAN | 18. | 18. | 18. | 18. |
| | S.D. | 1.9 | 2.2 | 1.9 | 1.1 |
| | N | 10 | 10 | 10 | 6 |
| | 7 TO 8 | | | | |
| | MEAN | 17 | 18. | 17. | 18. |
| | S.D. | 1.9 | 2.8 | 1.6 | 1.6 |
| | N | 10 | 10 | 10 | 6 |
| | 8 TO 9 | | | | |
| | MEAN | 18. | 19. | 19. | 18. |
| | S.D. | 2.3 | 2.6 | 2.0 | 1.2 |
| | N | 10 | 10 | 10 | 6 |
| WEEK | 9 TO 10 | | | | |
| | MEAN | 17. | 18. | 18. | 17. |
| | S.D. | 3.2 | 1.8 | 1.5 | 1.2 |
| | N | 10 | 10 | 10 | 6 |
| | 10 TO 11 | | | | |
| | MEAN | 17. | 18. | 18. | 17. |
| | S.D. | 2.6 | 1.9 | 1.6 | 2.3 |
| | N | 10 | 10 | 10 | 6 |
| | 11 TO 12 | | | | |
| | MEAN | 18. | 18. | 28. | 18. |
| | S.D. | 1.8 | 1.6 | 1.3 | 1.3 |
| | N | 10 | 10 | 10 | 6 |
| | 12 TO 13 | | | | |
| | MEAN | 18. | 17. | 17 | 17. |
| | S.D. | 2.5 | 2.3 | 3.7 | 1.0 |
| | N | 10 | 10 | 10 | 6 |

*= Significantly different from the control group at 0.05 using Dunnatt's test
**= Significantly different from the control group at 0.01 using Dunnatt's test

The rats exhibited higher alanine aminotransferase (ALT) and cholesterol levels and higher urine volume in both males and females, and 35% to 55% higher alkaline phosphatase (ALP) (males) and bilirubin (females) in the subjects of the 60 mg/kg/day group and occurred in the presence of microscopic observations of hepatocellular hypertrophy and vacuolation. Changes in cholesterol (both sexes) and urine volume (males only) were also found in the rats in the group receiving 25 mg/kg/day. Urine volume was approximately 90% to 170% higher than controls in the 60 mg/kg/day group males and females and 25 mg/kg/day group males. Dose-related changes in organ weights (absolute, relative to final body or brain weight) consisted of higher liver, kidney and thyroid weights and lower epididymis and uterine weights. Microscopic findings accompanied weight changes only in the liver and epididymis. Liver weights (relative to final body weight) were 7%, 22% and 43% higher than controls in 10, 25 and 60 mg/kg/day males, respectively, and 6%, 19% and 51% higher than

TABLE 6
Toxicokinetic Results
(+)-1-(3,4-dichlorophenyl)-3-azabicyclo[3, 1.0]hexane (free base) Results

| Gender/ | | | | | | |
|---|---|---|---|---|---|---|
| (mg/ | AUC₀₋₁₄ₕ (ng · h/mL) | | Cₘₐₓ (ng/mL) | | tₘₐₓ (h) | |
| kg/day) | Day 0 | Day 87 | Day 0 | Day 87 | Day 0 | Day 87 |

Males

| | | | | | | |
|---|---|---|---|---|---|---|
| 10 | 5940 | 8133 | 863 | 1116 | 2 | 1 |
| 25 | 17401 | 21975 | 2355 | 2486 | 2 | 2 |
| 60 | 36257 | 60093 | 3685 | 5013 | 1 | 1 |

Females

| | | | | | | |
|---|---|---|---|---|---|---|
| 10 | 10920 | 14350 | 1392 | 1848 | 1 | 1 |
| 25 | 30969 | 30205 | 2811 | 3702 | 2 | 2 |
| 60 | 54368 | 88137 | 6596 | 6529 | 2 | 2 |

controls in 10, 25 and 60 mg/kg/day females, respectively. Epididymis weights were 11% and 13% lower than control in the 25 and 60 mg/kg/day group males, respectively, accompanied by interstitial edema, subacute inflammation and/or tubular degeneration of the epididymis. Higher kidney weights relative to final body weights were observed in the 60 mg/kg/day group males and females (24% and 22% higher than control, respectively) and were associated with higher urea nitrogen (females) and higher urine volume (both sexes), but no histological changes. Mean thyroid weights (absolute, relative to final body weight or to brain weight) ranged from 24% to 27% and 37% to 45% higher than control values in the 25 and 60 mg/kg/day group females, respectively, in the absence of any histopathologic findings. Mean uterus weights (relative to final body weight) ranged from 23% to 50% lower than that of control in all treated groups. Based upon the magnitude of the weight change, this effect was considered test article-related, but there were no associated histopathologic findings. The toxicokinetic parameters for (+)-1-(3,4-dichlorophenyl)-3-azabicyclo[3.1.0]hexane hydrochloride (free base) are summarized in Table 6 below.

All dose groups were exposed to (+)-1-(3,4-dichlorophenyl)-3-azabicyclo[3.1.0]hexane hydrochloride. The exposures to (+)-1-(3,4-dichlorophenyl)-3-azabicyclo[3.1.0]hexane hydrochloride increased dose-dependently over the range of 10 to 60 mg/kg/day. Exposure to (+)-1-(3,4-dichlorophenyl)-3-azabicyclo[3.1.0]hexane hydrochloride tended to increase slightly with repeated dosing. Female rats had higher AUC₀₋₂₄ and Cmax values for (+)-1-(3,4-dichlorophenyl)-3-azabicyclo[3.1.0]hexane hydrochloride than male rats (differences up to 85%) in all dose groups.

Body weight and food consumption in rats treated with (+)-1-(3,4-dichlorophenyl)-3-azabicyclo[3.1.0]hexane hydrochloride decreased, and treatment-related liver (weight and histopathology), epididymal (weight and histopathology), kidney (weight), thyroid (weight) and uterus (weight) effects occurred at doses of 25 mg/kg/day and above. Dose-related hepatocellular vacuolation and hypertrophy were noted in all dose groups. However, the minimal hepatic findings in the 10 mg/kg/day group were not accompanied by changes in measured indicators of hepatic damage, other histopathologic changes or general measures of toxicity. Therefore, the no-observed-adverse-effect level (NOAEL) for oral administration of (+)-1-(3,4-dichlorophenyl)-3-azabicyclo[3.1.0]hexane hydrochloride to rats for 13 weeks was 10 mg/kg/day. Corresponding study day 87 AUC₀₋₂₄ values for the 10 mg/kg/day group males and females were 8133 and 14350 ng·h/mL, respectively.

### Example 7

### Toxicity Studies in Dogs for (+)-1-(3,4-dichlorophenyl)-3-azabicyclo[3.1.0]hexane hydrochloride

Eighteen male and eighteen female beagle dogs were received from Ridglan Farms, Mt. Horeb, Wis. (+)-1-(3,4-dichlorophenyl)-3-azabicyclo[3.1.0]hexane hydrochloride was administered orally via capsules to the dogs once daily, for a minimum of 91 days at dosage levels of 2.0, 6.0 and 20 mg/kg/day (Groups 2-4). A concurrent control group (Group 1) received empty capsules on a comparable regimen. Each group consisted of four males and four females. All animals/sex/group were scheduled for the primary necropsy at the end of the 13-week treatment period.

The animals were observed twice daily for mortality and moribundity. Clinical examinations were performed daily at the time of dosing and approximately 1-2 and approximately 3 hours following dose administration. Detailed physical examinations were performed weekly. Clinical pathology evaluations (hematology, serum chemistry and urinalysis) were performed prior to the initiation of dose administration (study week −1) and prior to the scheduled necropsy (study week 13). Individual body weights were recorded weekly, beginning approximately two weeks prior to test article administration (study week −2). Mean body weights and mean body weight changes were calculated for each corresponding interval. Final body weights (fasted) were recorded prior to the scheduled necropsy. Blood samples for toxicokinetic evaluation were collected from all dogs on study days 0 and 88 at 0, 1, 2, 4, 8 and 24 hours after dose administration. Complete necropsies were performed on all dogs, selected organs were weighed and selected tissues were examined microscopically from the control and high dose group animals and animals euthanized in extremis. Gross lesions were also examined from the 2.0 and 6.0 mg/kg/day groups.

(+)-1-(3,4-dichlorophenyl)-3-azabicyclo[3.1.0]hexane hydrochloride-related clinical observations consisted primarily of dilated pupils one and three hours following dose administration in all test article-treated groups. Additional (+)-1-(3,4-dichlorophenyl)-3-azabicyclo[3.1.0]hexane hydrochloride-related clinical findings consisted of reddened ears, emesis, wet clear material around the mouth and partial eyelid closure in the 6.0 and 20 mg/kg/day groups. These findings were attributed to the extended pharmacology of the test article. Increased post-dosing incidences of soft feces occurred primarily in the females of the 20 mg/kg/day group.

The toxicokinetic results are summarized in Table 7 below.

TABLE 7
Toxicokinetic Results
(+)-1-(3,4-dichlorophenyl)-3-azabicyclo[3, 1.0]hexane (free base) Results

| Gender/ | | | | | | |
|---|---|---|---|---|---|---|
| (mg/ | AUC_{0-24 h} (ng · h/mL) | | Cₘₐₓ (ng/mL) | | tₘₐₓ(h) | |
| kg/day) | Day 0 | Day 88 | Day 0 | Day 88 | Day 0 | Day 88 |

Males

| | | | | | | |
|---|---|---|---|---|---|---|
| 2 | 1570 | 2275 | 507 | 753 | 1 | 1 |
| 6 | 12836 | 13121 | 4637 | 4143 | 1 | 1 |
| 20 | 47843 | 44011 | 11392 | 7229 | 1 | 1 |

Females

| | | | | | | |
|---|---|---|---|---|---|---|
| 2 | 1476 | 2154 | 635 | 663 | 1 | 1 |
| 6 | 13568 | 15908 | 5119 | 4847 | 1 | 1 |
| 20 | 63607 | 29557 | 9278 | 8434 | 2 | 1 |

Administration of (+)-1-(3,4-dichlorophenyl)-3-azabicyclo[3.1.0]hexane hydrochloride reduced body weight gains and food consumption in the 20 mg/kg/day group throughout the study. Mean total cumulative body weight changes in the 20 mg/kg/day group males and females were 120% (body weight loss) and 65% lower, respectively, and by the end of the study, mean body weights of males and females were 13% and 9% lower, respectively, than the control group. These body weight decreases were accompanied by reduced food consumption (generally at least 10% less than control values in the males) throughout the study in this group.

Changes in organ weights consisted of higher mean liver weight relative to final body weight in the 20 mg/kg/day group males and females (19% and 27% higher, respectively). No macroscopic or microscopic changes accompanied the higher liver weights. No changes in hematology, serum chemistry, urinalysis, ophthalmic or electrocardiographic parameters were noted.

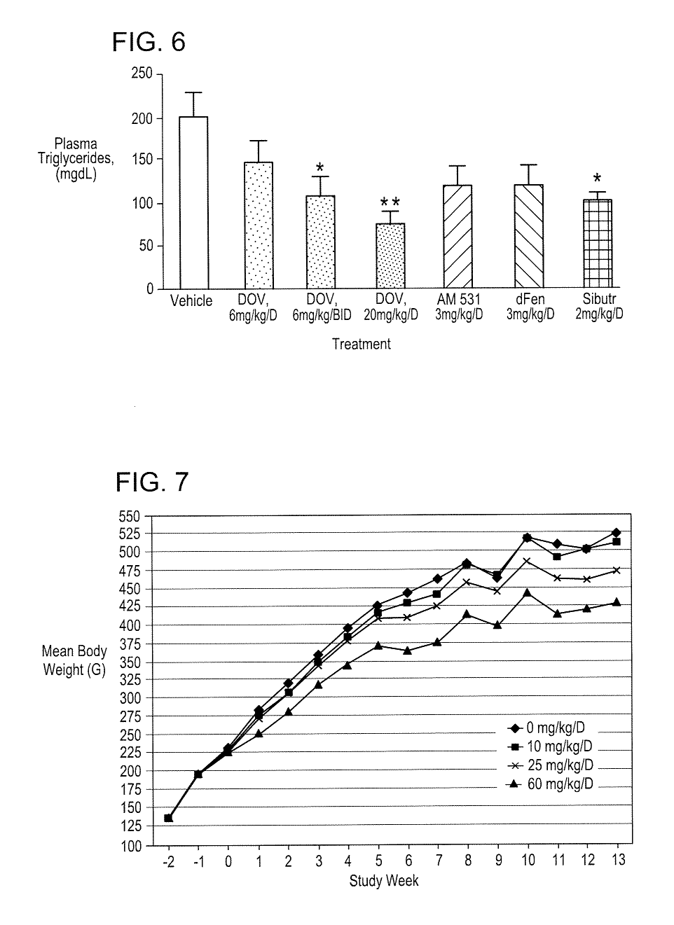

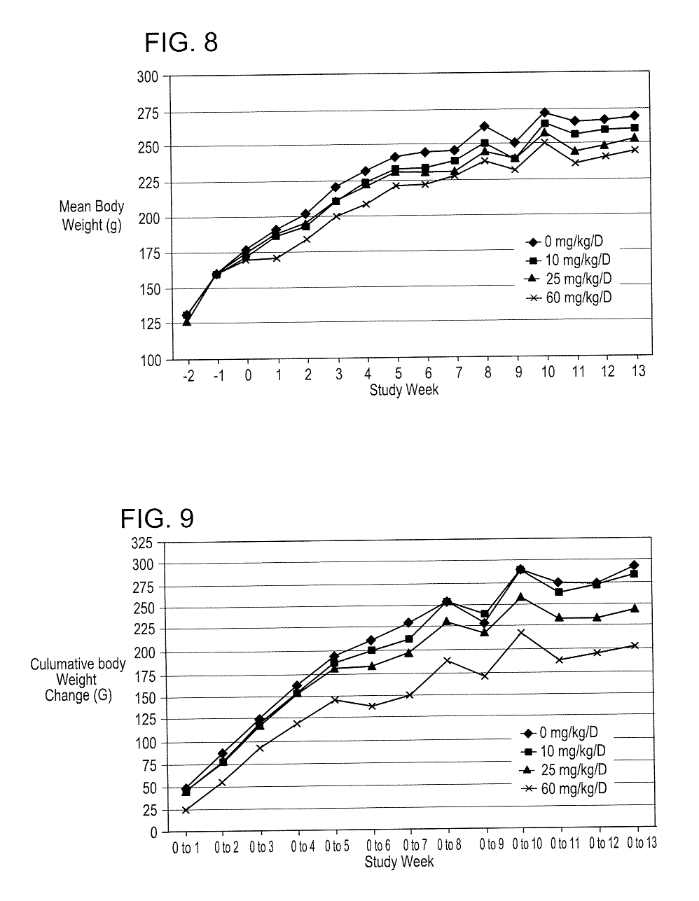

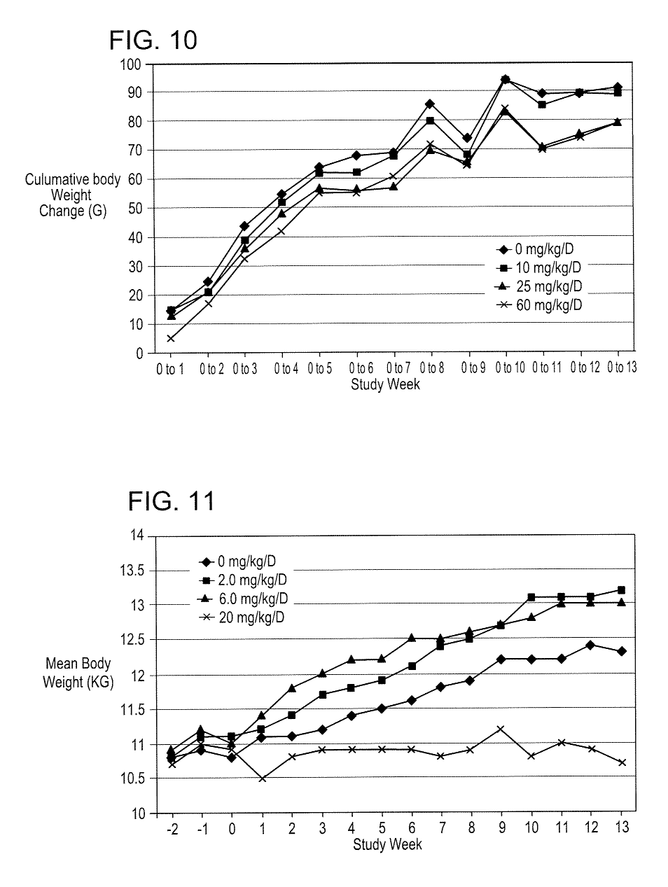

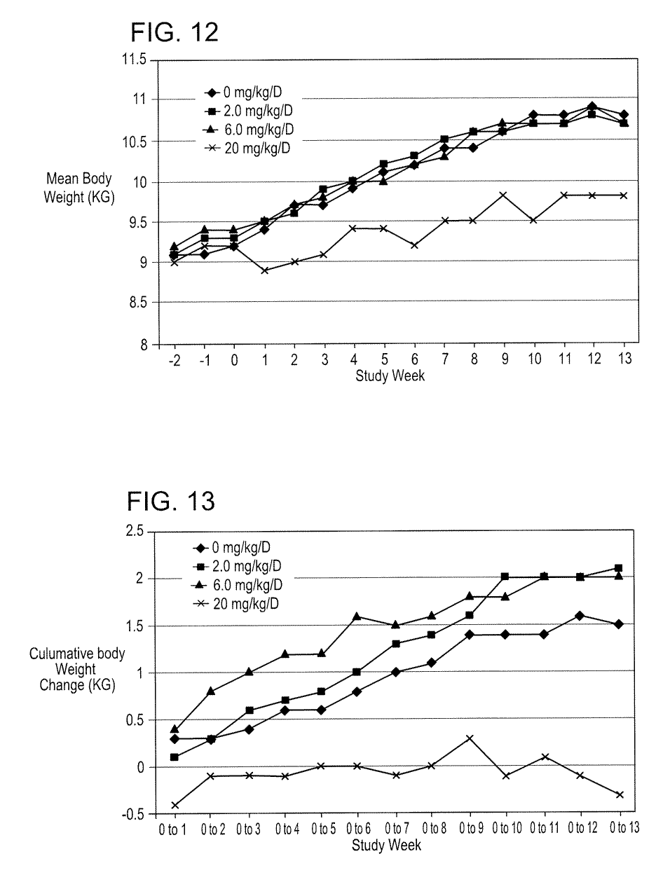

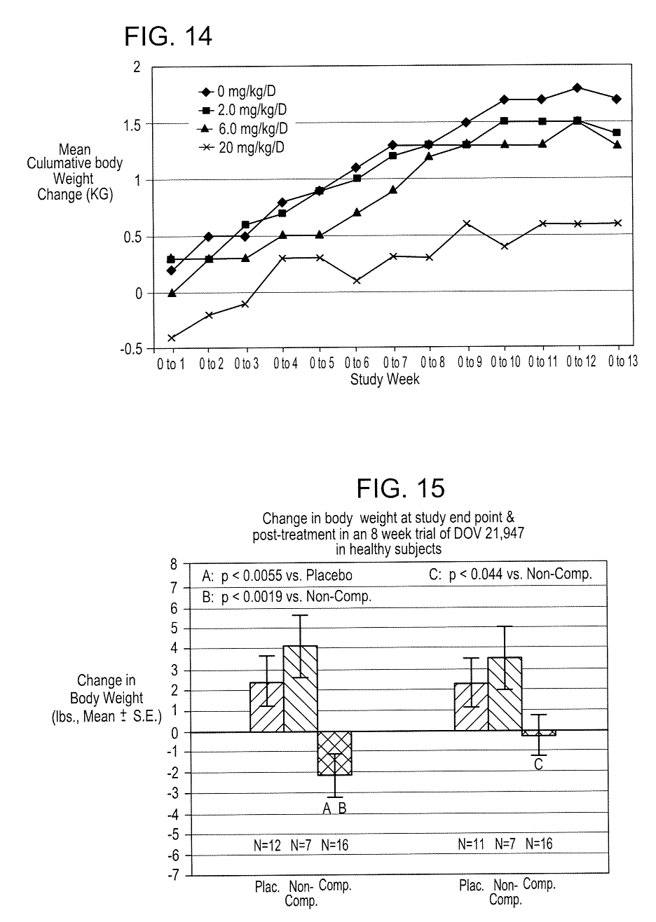

Reduced mean body weight changes (statistically significant at p<0.05 or p<0.01 when compared to the control group) resulted in a net body weight loss in the 20 mg/kg/day group males and females during the first week of dose administration (study week 0). Mean cumulative body weight changes were significantly (p<0.05 or p<0.01) lower in the 20 mg/kg/day group males and females throughout the study (FIGS. 8 and 9). Mean total weight gain in the 20 mg/kg/day group males and females was 120% (body weight loss) and 65% lower, respectively, than the control group value by the end of the study. As a result, mean body weights of males and females in this group were 13% and 9%, respectively, lower than the controls by study week 13 (FIGS. 6 and 7).

There were no other adverse test article-related effects on body weights. Mean body weight gains in the 6.0 mg/kg/day group males and the 20 mg/kg/day group females were significantly (p<0.05 or p<0.01) higher than the control values during study weeks 1 to 2 and 10 to 11, respectively. In addition, mean cumulative body weight gains in the 6.0 mg/kg/day group males were significantly (p<0.05) higher than the control values during study week intervals 0 to 3, 0 to 4 and 0 to 6. However, lower mean food consumption was noted in the 20 mg/kg/day group. During the first week of dose administration, mean food consumption in the 20 mg/kg/day group was significantly (p<0.05) lower than the control group (33% and 23% for males (Table 8) and females (Table 9), respectively). Although not statistically significant, mean food consumption in the 20 mg/kg/day group males was at least 10% lower than the control group during study weeks 6 to 13, with the exception of study week 8 to 9.

TABLE 8
SUMMARY OF WEEKLY FOOD CONSUMPTION (G/ANIMAL/DAY)

| | | ----- M A L E ----- | | | |
|---|---|---|---|---|---|
| | GROUP: | 0 MG/KG/DAY | 2.0 MG/KG/DAY | 6.0 MG/KG/DAY | 20 MG/KG/DAY |
| WEEK | −1 TO 0 | | | | |
| | MEAN | 340. | 330. | 331. | 337. |
| | S.D. | 50.5 | 40.7 | 23.6 | 43.6 |
| | N | 4 | 4 | 4 | 4 |
| | 0 TO 1 | | | | |
| | MEAN | 342. | 320. | 351. | 228.* |
| | S.D. | 54.0 | 66.3 | 32.5 | 55.2 |
| | N | 4 | 4 | 4 | 4 |
| | 1 TO 2 | | | | |
| | MEAN | 352. | 354. | 370. | 329. |
| | S.D. | 51.1 | 50.2 | 28.0 | 60.2 |
| | N | 4 | 4 | 4 | 4 |
| | 2 TO 3 | | | | |
| | MEAN | 380. | 373. | 376. | 326. |
| | S.D. | 33.0 | 30.6 | 16.0 | 34.0 |
| | N | 4 | 4 | 4 | 4 |
| | 3 TO 4 | | | | |
| | MEAN | 383. | 366. | 380. | 357. |
| | S.D. | 21.5 | 43.1 | 20.0 | 30.0 |
| | N | 4 | 4 | 4 | 4 |
| WEEK | 4 TO 5 | | | | |
| | MEAN | 374. | 369. | 350. | 356. |
| | S.D. | 45.7 | 40.3 | 45.0 | 42.2 |
| | N | 4 | 4 | 4 | 4 |
| | 5 TO 6 | | | | |
| | MEAN | 363. | 360. | 374. | 331. |
| | S.D. | 78.3 | 47.7 | 25.3 | 28.5 |
| | N | 4 | 4 | 4 | 4 |
| | 6 TO 7 | | | | |
| | MEAN | 382. | 393. | 381. | 128. |
| | S.D. | 39.2 | 13.5 | 36.6 | 100.1 |
| | N | 4 | 4 | 4 | 4 |
| | 7 TO 8 | | | | |
| | MEAN | 374. | 360. | 356. | 325. |
| | S.D. | 26.6 | 47.5 | 66.2 | 18.2 |
| | N | 4 | 4 | 4 | 4 |
| | 8 TO 9 | | | | |
| | MEAN | 384. | 388. | 368. | 375. |
| | S.D. | 35.8 | 23.0 | 41.5 | 17.9 |
| | N | 4 | 4 | 4 | 4 |
| WEEK | 9 TO 10 | | | | |
| | MEAN | 390. | 375. | 360. | 322. |
| | S.D. | 21.0 | 20.4 | 53.2 | 36.0 |
| | N | 4 | 4 | 4 | 4 |
| | 10 TO 11 | | | | |
| | MEAN | 394. | 389. | 367. | 350. |
| | S.D. | 15.8 | 15.8 | 30.0 | 60.2 |
| | N | 4 | 4 | 4 | 4 |
| | 11 TO 12 | | | | |
| | MEAN | 386. | 378. | 357. | 305. |
| | S.D. | 32.0 | 36.8 | 54.8 | 29.3 |
| | N | 4 | 4 | 4 | 4 |
| | 12 TO 13 | | | | |
| | MEAN | 357. | 355. | 331. | 249. |
| | S.D. | 54.6 | 26.3 | 42.9 | 93.6 |
| | N | 4 | 4 | 4 | 4 |

*= Significantly different from the control group at 0.05 using Dunnatt's test

TABLE 9
SUMMARY OF WEEKLY FOOD CONSUMPTION (G/ANIMAL/DAY)

| | | ----- F E M A L E ----- | | | |
|---|---|---|---|---|---|
| | GROUP: | 0 MG/KG/DAY | 2.0 MG/KG/DAY | 6.0 MG/KG/DAY | 20 MG/KG/DAY |
| WEEK | −1 TO 0 | | | | |
| | MEAN | 270. | 295. | 275. | 275. |
| | S.D. | 28.7 | 12.2 | 42.6 | 7.0 |
| | N | 4 | 4 | 4 | 4 |
| | 0 TO 1 | | | | |
| | MEAN | 275. | 201. | 247. | 212.* |
| | S.D. | 20.7 | 4.6 | 9.4 | 61.0 |
| | N | 4 | 4 | 4 | 4 |
| | 1 TO 2 | | | | |
| | MEAN | 306. | 319. | 288. | 295. |
| | S.D. | 22.9 | 23.6 | 22.1 | 40.9 |
| | N | 4 | 4 | 4 | 4 |
| | 2 TO 3 | | | | |
| | MEAN | 336. | 361. | 307. | 314. |
| | S.D. | 20.8 | 38.7 | 10.7 | 62.0 |
| | N | 4 | 4 | 4 | 4 |
| | 3 TO 4 | | | | |
| | MEAN | 353. | 358. | 307. | 337. |
| | S.D. | 11.4 | 34.6 | 6.7 | 50.8 |
| | N | 4 | 4 | 4 | 4 |
| WEEK | 4 TO 5 | | | | |
| | MEAN | 374. | 369. | 350. | 356. |
| | S.D. | 45.7 | 40.3 | 45.0 | 42.2 |
| | N | 4 | 4 | 4 | 4 |
| | 5 TO 6 | | | | |
| | MEAN | 363. | 360. | 374. | 331. |
| | S.D. | 78.3 | 47.7 | 25.3 | 28.5 |
| | N | 4 | 4 | 4 | 4 |
| | 6 TO 7 | | | | |
| | MEAN | 382. | 393. | 381. | 328. |
| | S.D. | 39.2 | 13.5 | 36.6 | 100.1 |
| | N | 4 | 4 | 4 | 4 |
| | 7 TO 8 | | | | |
| | MEAN | 374. | 360. | 356. | 325. |
| | S.D. | 26.6 | 47.5 | 66.2 | 18.2 |
| | N | 4 | 4 | 4 | 4 |
| | 8 TO 9 | | | | |
| | MEAN | 384. | 388. | 368. | 375. |
| | S.D. | 35.8 | 23.0 | 41.5 | 17.9 |
| | N | 4 | 4 | 4 | 4 |
| WEEK | 9 TO 10 | | | | |
| | MEAN | 390. | 375. | 360. | 322. |
| | S.D. | 21.9 | 29.4 | 53.2 | 36.0 |
| | N | 4 | 4 | 4 | 4 |
| | 10 TO 11 | | | | |
| | MEAN | 394. | 389. | 367. | 350. |
| | S.D. | 15.8 | 15.8 | 30.0 | 69.2 |
| | N | 4 | 4 | 4 | 4 |
| | 11 TO 12 | | | | |
| | MEAN | 386. | 378. | 357. | 305. |
| | S.D. | 32.0 | 36.8 | 54.8 | 29.3 |
| | N | 4 | 4 | 4 | 4 |
| | 12 TO 13 | | | | |
| | MEAN | 357. | 355. | 331. | 249. |
| | S.D. | 54.6 | 26.3 | 42.9 | 93.6 |
| | N | 4 | 4 | 4 | 4 |

*= Significantly different from the control group at 0.05 using Dunnatt's test

Based on body weight loss and/or lower body weight gains, reduced food consumption and increased relative liver weights at 20 mg/kg/day, the no-observed-adverse-effect level (NOAEL) for oral (capsule) administration of (+)-1-(3,4-dichlorophenyl)-3-azabicyclo[3.1.0]hexane to dogs for 13 weeks was 6.0 mg/kg/day. Corresponding study day 88 AUC₀₋₂₄ values for the 6.0 mg/kg/day group males and females were 13121 and 15908 ng·h/mL, respectively.

### Example 8

### Effects of (+)-1-(3,4-Dichlorophenyl)-3-azabicyclo[3.1.0]hexane hydrochloride (DOV 21947) on body weight, body mass index and plasma triglycerides in human subjects

A multiple-dose, randomized, double-blind, placebo-controlled safety and tolerability study of (+)-1-(3,4-Dichlorophenyl)-3-azabicyclo[3.1.0]hexane hydrochloride (DOV 21947) was performed on healthy subjects with a body mass index (BMI) >25 and <35 (i.e., overweight to moderately obese individuals). One of the goals of this study was to explore the efficacy of (+)-1-(3,4-Dichlorophenyl)-3-azabicyclo[3.1.0]hexane hydrochloride (DOV 21947) to induce weight loss.

Subjects were screened for suitability to participate in this trial. After completing the screening assessments, a total of 45 male and female subjects with BMI ≧25 to ≦35 were randomized in a ratio of 2:1 into 2 groups to receive double-blind BID treatment with either (+)-1-(3,4-Dichlorophenyl)-3-azabicyclo[3.1.0]hexane hydrochloride (DOV 21947) titrated from 25 mg BID for 2 weeks, to 50 mg BID for 2 weeks, to 75 mg BID for 4 weeks, or Placebo. Blood samples were taken at baseline, and every week throughout the study for the determination of clinical chemistry (particularly, triglyceride levels) and the levels of (+)-1-(3,4-Dichlorophenyl)-3-azabicyclo[3.1.0]hexane hydrochloride (DOV 21947), in order to assure compliance. On the day of the last dose in the study, blood was taken for triglyceride measures, as well as the body weight and height of the subject. The latter two factors were used to determine the body mass index (BMI).

FIG. 15 indicates that individuals who adhered to the (+)-1-(3,4-Dichlorophenyl)-3-azabicyclo[3.1.0]hexane hydrochloride (DOV 21947) treatment regimen (compliant group) showed an average weight loss of 2 pounds on the last day of treatment compared to their baseline weight (left side), whereas non-compliant and placebo treated subjects showed no significant weight loss relative to baseline weight. By 7 days after discontinuation of the drug, the body weight of the (+)-1-(3,4-Dichlorophenyl)-3-azabicyclo[3.1.0]hexane hydrochloride (DOV 21947) compliant group had returned to baseline levels.

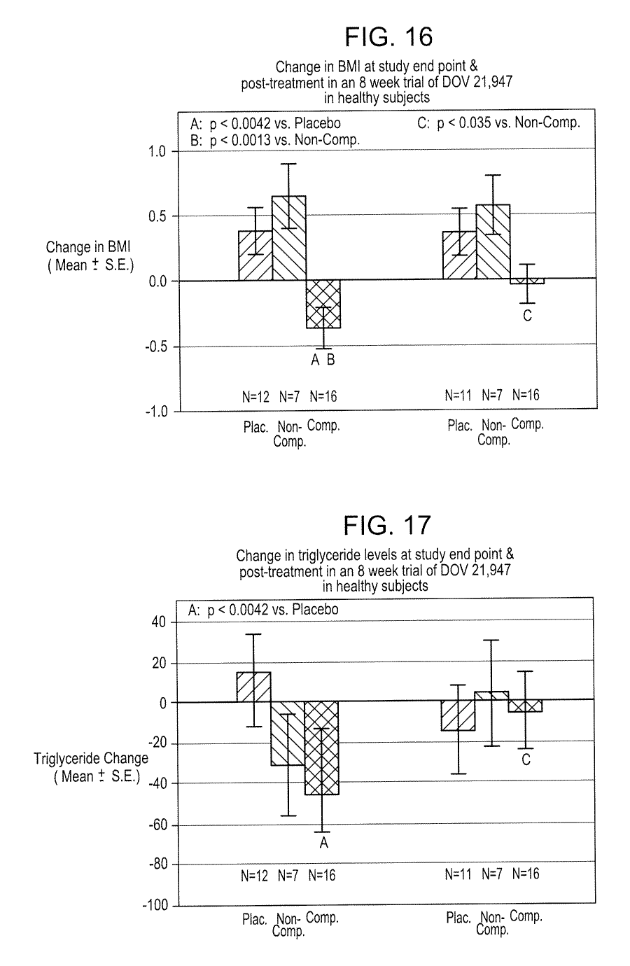

FIG. 16 indicates that individuals who adhered to the (+)-1-(3,4-Dichlorophenyl)-3-azabicyclo[3.1.0]hexane hydrochloride (DOV 21947) treatment regimen (compliant group) showed a significant decrease in BMI as of the last day of treatment compared to their baseline BMI (left side), whereas non-compliant and placebo treated subjects showed no significant decrease in BMI relative to baseline. By 7 days after discontinuation of the drug, the BMI of the (+)-1-(3,4-Dichlorophenyl)-3-azabicyclo[3.1.0]hexane hydrochloride (DOV 21947) compliant group had returned to baseline levels.

FIG. 17 indicates that individuals who adhered to the (+)-1-(3,4-Dichlorophenyl)-3-azabicyclo[3.1.0]hexane hydrochloride (DOV 21947) treatment regimen (compliant group) showed a significant decrease in plasma triglyceride levels as of the last day of treatment compared to their baseline levels (left side), whereas non-compliant and placebo treated subjects showed no significant decrease in triglyceride levels relative to baseline. By 7 days after discontinuation of the drug, the triglyceride levels of the (+)-1-(3,4-Dichlorophenyl)-3-azabicyclo[3.1.0]hexane hydrochloride (DOV 21947) compliant group had returned to baseline levels.

In summary, these data indicate that (+)-1-(3,4-Dichlorophenyl)-3-azabicyclo[3.1.0]hexane hydrochloride (DOV 21947) is a potent inhibitor of DA, NE and 5-HT uptake, and can cause significant weight loss selective for fat mass, as well as a significant reduction in plasma triglyceride levels in animal models of obesity. This decrease in body mass is sustained for the duration of (+)-1-(3,4-Dichlorophenyl)-3-azabicyclo[3.1.0]hexane hydrochloride (DOV 21947) administration, and is reversible upon cessation of treatment. Moreover, the results from the DIO rats are predictive of the human condition, in that overweight subjects treated with (+)-1-(3,4-Dichlorophenyl)-3-azabicyclo[3.1.0]hexane hydrochloride (DOV 21947) manifested a significant decrease in body weight and plasma triglyceride levels.

Although the foregoing invention has been described in detail by way of example for purposes of clarity of understanding, it will be apparent to the artisan that certain changes and modifications may be practiced within the scope of the appended claims which are presented by way of illustration not limitation. In this context it will be understood that this invention is not limited to the particular formulations, process steps, and materials disclosed herein as such formulations, process steps, and materials may vary somewhat. It will also be understood that the terminology employed herein is used for the purpose of describing particular embodiments only, and is not intended to be limiting since the scope of the present invention will be limited only by the appended claims and equivalents thereof. It is further noted that various publications and other reference information have been cited within the foregoing disclosure for economy of description. Each of these references are incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

1. A method of treating or preventing obesity in a mammalian subject comprising
administering an effective amount of a (+)-1-(3,4-dichlorophenyl)-3-azabicyclo[3.1.0]hexane to said subject.

2. The method of claim 1, wherein the (+)-1-(3,4-dichlorophenyl)-3-azabicyclo[3.1.0]hexane is a pharmaceutically acceptable salt, polymorph, solvate, hydrate and/or prodrug thereof.

3. The method of claim 1, further comprising administering a second therapeutic agent to said subject.

4. The method of claim 3, wherein the second therapeutic agent is administered to said subject in a combined formulation with a (+)-1-(3,4-dichlorophenyl)-3-azabicyclo[3.1.0]hexane.

5. The method of claim 3, wherein said second therapeutic agent is administered to said subject in a coordinate administration protocol, simultaneously with, prior to, or after administration of said (+)-1-(3,4-dichlorophenyl)-3-azabicyclo[3.1.0]hexane to said subject.

6. The method of claim 3, wherein the second therapeutic agent is selected from insulin sensitizers, biguanides, protein tyrosine phosphatase-1B (PTP-1B) inhibitors, dipeptidyl peptidase IV (DP-IV) inhibitors, insulin, insulin mimetics, sulfonylureas, α-glucosidase inhibitors, cholesterol lowering agents, sequestrants, nicotinyl alcohol, nicotinic acid or a salt thereof, PPARα agonists, PPARα/γ dual agonists, anti-obesity compounds, inhibitors of cholesterol absorption, acyl CoA:cholesterol acyltransferase inhibitors, anti-oxidants, neuropeptide Y5 inhibitors, β₃ adrenergic receptor agonists, an ileal bile acid transporter inhibitor, a non-steroidal anti-inflammatory drugs, glucocorticoids, azulfidine, or cyclo-oxygenase 2 selective inhibitors.

7. The method of claim 6, wherein the PPARγ agonists are glitazones.

8. The method of claim 6, wherein the biguanides are metformin or phenformin.

9. The method of claim 6, wherein the sulfonylureas are tolbutamide or glipizide.

10. The method of claim 6, wherein the cholesterol lowering agents are lovastatin, simvastatin, pravastatin, fluvastatin, atorvastatin, rivastatin, itavastatin, or ZD-4522.

11. The method of claim 6, wherein the sequestrant is cholestyramine, colestipol, or dialkylaminoalkyl derivatives of a cross-linked dextran.

12. The method of claim 6, wherein the PPARα agonists is gemfibrozil, clofibrate, fenofibrate or bezafibrate.

13. The method of claim 6, wherein the anti-obesity compounds is fenfluramine, dexfenfluramine, phentiramine, sulbitramine, diethylpropion, adderall, mazindol, benzphetamine, or orlistat.

14. The method of claim 1, further comprising an anti-obesity physical treatment.

15. The method of claim 14, wherein the anti-obesity physical treatment is diet, psychological counseling, behavior modification, exercise or surgery.

16. The method of claim 15, wherein the surgery is gastric partitioning procedures, jejunoileal bypass, stomach stapling, gastric bands, vertical banded gastroplasty, laparoscopic gastric banding, roux-en-Y gastric bypass, biliopancreatic bypass procedures or vagotomy.

17. The method of claim 1, wherein the effective amount comprises between about 0.01 mg to about 100 mg of a (+)-1-(3,4-dichlorophenyl)-3-azabicyclo[3.1.0]hexane per kg per day.

18. The method of claim 1, wherein the effective amount comprises between about 0.1 mg to about 75 mg of a (+)-1-(3,4-dichlorophenyl)-3-azabicyclo[3.1.0]hexane per kg per day.

19. The method of claim 1, wherein the effective amount comprises between about 0.5 mg to about 50 mg of a (+)-1-(3,4-dichlorophenyl)-3-azabicyclo[3.1.0]hexane per kg per day.

20. The method of claim 1, wherein the effective amount comprises between about 1 mg to about 40 mg of a (+)-1-(3,4-dichlorophenyl)-3-azabicyclo[3.1.0]hexane per kg per day.

21. The method of claim 1, wherein the effective amount comprises between about 1 mg to 3 mg of a (+)-1-(3,4-dichlorophenyl)-3-azabicyclo[3.1.0]hexane per kg per day.

22. The method of claim 1, wherein the administration of (+)-1-(3,4-dichlorophenyl)-3-azabicyclo[3.1.0]hexane is effective to decrease body mass index to between about 18 kg/m² to about 30 kg/m².

23. The method of claim 1, wherein the administration of (+)-1-(3,4-dichlorophenyl)-3-azabicyclo[3.1.0]hexane is effective to decrease body mass index to between about 18 kg/m² to about 25 kg/m².

24. The method of claim 1, wherein the administration of (+)-1-(3,4-dichlorophenyl)-3-azabicyclo[3.1.0]hexane is effective to decrease body weight by about 5-50%.

25. The method of claim 1, wherein the administration of (+)-1-(3,4-dichlorophenyl)-3-azabicyclo[3.1.0]hexane is effective to decrease body weight by about 15-30%.

26. The method of claim 1, wherein the administration of (+)-1-(3,4-dichlorophenyl)-3-azabicyclo[3.1.0]hexane is effective to decrease body fat by about 5-50%.

27. The method of claim 1, wherein the administration of (+)-1-(3,4-dichlorophenyl)-3-azabicyclo[3.1.0]hexane is effective to decrease body fat by about 15-30%.

28. A method of preventing or alleviating complications associated with obesity in a mammalian subject comprising administering an effective amount of a of (+)-1-(3,4-dichlorophenyl)-3-azabicyclo[3.1.0]hexane to said subject.

29. The method of claim 28, wherein the complications are coronary heart disease, osteoarthritis, osteoporosis, dislipidemias, gout, atherosclerosis, joint pain, sexual and fertility problems, respiratory problems, gall bladder disease, skin conditions, hypertension, diabetes, stroke, pulmonary embolism, sleep apnea, idiopathic intracranial hypertension, lower extremity venous stasis disease, gastro-esophageal reflux, urinary stress incontinence, metabolic syndrome, insulin resistance and cancer.

30-54. (canceled)

55. A method for reducing appetite or caloric intake in a mammalian subject comprising administering an effective amount of a (+)-1-(3,4-dichlorophenyl)-3-azabicyclo[3.1.0]hexane to said subject.

56-81. (canceled)
